(19) United States
(12) Patent Application Publication
(10) Pub. No.: US 2016/0289259 A1
(43) Pub. Date: Oct. 6, 2016

(54) 2'-Branched Nucleosides for Treatment of Viral Infections

(71) Applicant: Fumiaki Yokokawa, Singapore (SG)

(72) Inventor: Fumiaki Yokokawa, Singapore (SG)

(73) Assignee: Novartis AG, Basel (CH)

(21) Appl. No.: 15/184,938

(22) Filed: Jun. 16, 2016

Related U.S. Application Data
(63) Continuation of application No. 14/011,500, filed on Aug. 27, 2013, now abandoned.
(60) Provisional application No. 61/695,727, filed on Aug. 31, 2012.

(51) Int. Cl. C07H 19/10; A61K 45/06; A61K 31/7072

(52) U.S. Cl. CPC C07H 19/10; A61K 31/7072; A61K 45/06

(57) ABSTRACT

The present invention provides a compound of formula I:

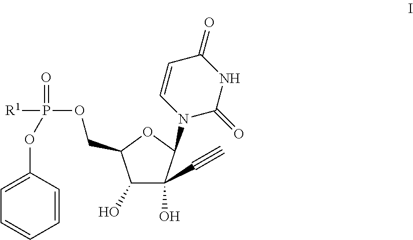

or a pharmaceutically acceptable salt thereof, wherein R¹ is defined herein, which is a 2′-branched nucleoside useful for the treatment or prevention of viral infections, particularly dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus.

### CROSS-REFERENCE TO RELATED APPLICATION

This application is continuation of U.S. Ser. No. 14/011,500, filed Aug. 27, 2013, which claims the benefit of priority under 35 U.S.C. §119(e) to U.S. provisional application Ser. No. 61/695,727, filed on Aug. 31, 2012, each of which is incorporated herein in its entirety by reference.

### FIELD OF THE INVENTION

This invention is directed to novel compounds which are useful in the treatment of viral infections. The invention is also directed to pharmaceutical compositions containing the compounds, processes for their preparation and uses of the compounds in various medicinal applications, such as the treatment or prevention of viral infections, particularly dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus, more particularly dengue virus and Hepatitis C virus.

### BACKGROUND

Dengue fever is a febrile disease caused by one of the four dengue virus serotypes DEN-1, DEN-2, DEN-3 and DEN-4, which belong to the family Flaviviridae. The virus is transmitted to humans primarily by Aedes aegypti, a mosquito that feeds on humans.

Infections produce a range of clinical manifestations, from milder flu-like symptoms to the more severe and sometimes fatal hemorrhagic disease. Typical symptoms include fever, severe headache, muscle and joint pains and rashes. The more severe forms of the disease are dengue hemorrhagic fever (DHF) and dengue shock syndrome (DSS). According to the WHO, there are four major clinical manifestations of DHF: (1) high fever (2) haemorrhagic phenomena (3) thrombocytopaenia and (4) leakage of plasma. DSS is defined as DHF plus weak rapid pulse, and narrow pulse pressure or hypotension with cold, clammy skin and restlessness. The severity of DHF can be reduced with early detection and intervention, but subjects in shock are at high risk of death.

Dengue is endemic in tropical regions, particularly in Asia, Africa and Latin America, and an estimated 2.5 billion people live in areas where they are at risk of infection. There are around 40 million cases of dengue fever and several hundred thousand cases of DHF each year. In Singapore, an epidemic in 2005 resulted in more than 12000 cases of dengue fever.

Despite regular outbreaks, previously infected people remain susceptible to infection because there are four different serotypes of the dengue virus and infection with one of these serotypes provides immunity to only that serotype. It is believed that DHF is more likely to occur in subjects who have secondary dengue infections. Efficient treatments for dengue fever, DHF and DSS are being sought.

Yellow fever virus (YFV), West Nile virus (WNV), Japanese encephalitis virus (JEV), tick-borne encephalitis virus, Kunjin virus, Murray Valley encephalitis, St Louis encephalitis, Omsk hemorrhagic fever virus, bovine viral diarrhea virus, Zika virus and Hepatitis C virus (HCV) also belong to the family Flaviviridae.

WNV can be asymptomatic, or it can cause flu-like symptoms in some individuals. In some cases it causes neurological disorders, encephalitis, and in severe cases can result in death. WNV is also transmitted by mosquitos. YFV is another mosquito-borne virus, which can cause severe symptoms in infected individuals. JEV is also transmitted by mosquitoes, and is either asymptomatic or causes flu-like symptoms, with some cases developing into encephalitis. The acute encephalitis stage of the disease is characterised by convulsions, neck stiffness and other symptoms.

HCV is a blood-borne virus that is transmitted by blood-to-blood contact. In the initial (acute) stage of the disease, most subjects will not show any symptoms. Even during the chronic stage (i.e. where the disease persists for more than 6 months), severity of symptoms can vary from subject to subject. In the long term, some infected persons can progress to cirrhosis and liver cancer. The current treatment for HCV involves a combination of interferon alpha and ribavirin, an anti-viral drug.

Efficient treatments for infections caused by these Flaviviridae viruses are being sought as well.

It has now surprisingly been found that certain nucleoside analogs are useful for the treatment of viral infections such as those caused by a virus of the family Flaviviridae, especially dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus, and other Flaviviridae viruses as described herein.

### SUMMARY

The invention provides compounds and pharmaceutical compositions thereof, which are useful for the treatment of viral infections.

In a first embodiment, the invention provides a compound of formula (I), or a pharmaceutically acceptable salt thereof:

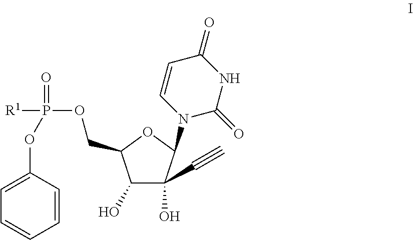

wherein

R¹ is

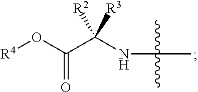

R² is a C₁-C₆ alkyl optionally substituted with halogen, a C₃-C₇ cycloalkyl optionally substituted with halogen, a phenyl optionally substituted with halogen or C₁-C₄alkyl or a C₁-C₄alkyl-phenyl optionally substituted with halogen or C₁-C₄alkyl;

R³ is H or C₁-C₄ alkyl

R² and R³ taken together and the carbon atom they are attached form a C₃-C₇ cycloalkyl;

R⁴ is C₁-C₈ alkyl optionally substituted with halogen or C₁-C₄alkoxy, a C₃-C₇ cycloalkyl optionally substituted with halogen, a phenyl optionally substituted with halogen or C₁-C₄alkyl; a C₁-C₄alkyl-phenyl optionally substituted with halogen or C₁-C₄alkyl or a 4 to 7 membered heterocycle containing 1 to 3 heteroatom selected from N, S, and O, wherein said heterocycle is optionally substituted with one or more halogen, or C₁-C₄ alkyl.

### DEFINITIONS

For purposes of interpreting this specification, the following definitions will apply unless specified otherwise and whenever appropriate, terms used in the singular will also include the plural and vice versa.

### DEFINITIONS

Terms used in the specification have the following meanings:

“Optionally substituted” means the group referred to can be substituted at one or more positions by any one or any combination of the radicals listed thereafter.

“Halo” or “halogen”, as used herein, may be fluorine, chlorine, bromine or iodine.

“C₁-C₆-Alkyl”, as used herein, denotes straight chain or branched alkyl having 1-8 carbon atoms. If a different number of carbon atoms is specified, such as C₆ or C₃, then the definition is to be amended accordingly, such as “C₁-C₄-Alkyl” will represent methyl, ethyl, propyl, isopropyl, butyl, isobutyl, sec-butyl and tert-butyl.

“C₁-C₆-Alkoxy”, as used herein, denotes straight chain or branched alkoxy having 1-8 carbon atoms. If a different number of carbon atoms is specified, such as C₆ or C₃, then the edefinition is to be amended accordingly, such as “C₁-C₄-Alkoxy” will represent methoxy, ethoxy, propoxy, isopropoxy, butoxy, isobutoxy, sec-butoxy and tert-butoxy.

“C₁-C₄-Haloalkyl”, as used herein, denotes straight chain or branched alkyl having 1-4 carbon atoms with at least one hydrogen substituted with a halogen. If a different number of carbon atoms is specified, such as C₆ or C₃, then the definition is to be amended accordingly, such as “C₁-C₄-Haloalkyl” will represent methyl, ethyl, propyl, isopropyl, butyl, isobutyl, sec-butyl and tert-butyl that have at least one hydrogen substituted with halogen, such as where the halogen is fluorine: CF₃CF₂—, (CF₃)₂CH—, CH₃—CF₂—, CF₃CF₂—, CF₃, CF₂H—, CF₃CF₂CHCF₃ or CF₃CF₂CF₂CF₂—.

“C₃-C₈-cycloalkyl” as used herein refers to a saturated monocyclic hydrocarbon ring of 3 to 8 carbon atoms. Examples of such groups include cyclopropyl, cyclobutyl, cyclopentyl and cyclohexyl. If a different number of carbon atoms is specified, such as C₃-C₆, then the definition is to be amended accordingly.

“aryl” or “C₆-C₁₅-aryl”, as used herein, denotes an aromatic group having 6- to 15-ring carbon atoms. Examples of C₆-C₁₅-aryl groups include, but are not limited to, phenyl, phenylene, benzenetriyl, naphthyl, naphthylene, naphthalenetriyl or anthrylene. If a different number of carbon atoms is specified, such as C₁₀, then the definition is to be amended accordingly.

“4- to 8-Membered heterocyclyl”, “5- to 6-membered heterocyclyl”, “3- to 10-membered heterocyclyl”, “3- to 14-membered heterocyclyl”, “4- to 14-membered heterocyclyl” and “5- to 14-membered heterocyclyl”, refers, respectively, to 4- to 8-membered, 5- to 6-membered, 3- to 10-membered, 3- to 14-membered, 4- to 14-membered and 5- to 14-membered heterocyclic rings containing 1 to 7, 1 to 5 or 1 to 3 heteroatoms selected from the group consisting of nitrogen, oxygen and sulphur, which may be saturated, or partially saturated. The heterocyclic group can be attached at a heteroatom or a carbon atom. The term “heterocyclyl” includes single ring groups, fused ring groups and bridged groups. Examples of such heterocyclyl include, but are not limited to pyrrolidine, piperidine, piperazine, pyrrolidine, pyrrolidinone, morpholine, tetrahydrofuran, tetrahydrothiophene, tetrahydrothiopyran, tetrahydropyran, 1,4-dioxane, 1,4-oxathiane, 8-aza-bicyclo[3.2.1]octane, 3,8-diazabicyclo[3.2.1]octane, 3-Oxa-8-aza-bicyclo[3.2.1]octane, 8-Oxa-3-aza-bicyclo[3.2.1]octane, 2-Oxa-5-aza-bicyclo[2.2.1]heptane, 2,5-Diaza-bicyclo[2.2.1]heptane, azetidine, ethylenedioxo, oxtane or thiazole.

The term “a,” “an,” “the” and similar terms used in the context of the present invention (especially in the context of the claims) are to be construed to cover both the singular and plural unless otherwise indicated herein or clearly contradicted by the context.

Various embodiments of the invention are described herein. It will be recognized that features specified in each embodiment may be combined with other specified features to provide further embodiments.

In another embodiment, the invention provides a compound of formula (II) or a pharmaceutically acceptable salt thereof:

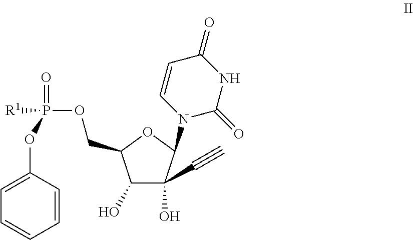

R¹ is

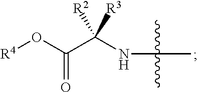

R² is a C₁-C₆ alkyl optionally substituted with halogen, a C₃-C₇ cycloalkyl optionally substituted with halogen, a phenyl optionally substituted with halogen or C₁-C₄alkyl or a C₁-C₄alkyl-phenyl optionally substituted with halogen or C₁-C₄alkyl;

R³ is H or C₁-C₄ alkyl

R² and R³ taken together and the carbon atom they are attached form a C₃-C₇ cycloalkyl;

R⁴ is C₁-C₈ alkyl optionally substituted with halogen or C₁-C₄alkoxy, a C₃-C₇ cycloalkyl optionally substituted with halogen, a phenyl optionally substituted with halogen or C₁-C₄alkyl; a C₁-C₄alkyl-phenyl optionally substituted with halogen or C₁-C₄alkyl or a 4 to 7 membered heterocycle containing 1 to 3 heteroatom selected from N, S, and O, wherein said heterocycle is optionally substituted with one or more halogen, or C₁-C₄ alkyl.

In another embodiment, the invention provides a compound of formula (III) or a pharmaceutically acceptable salt thereof:

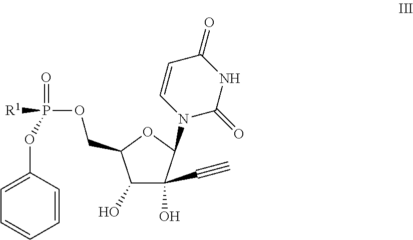

wherein,

R¹ is

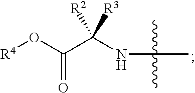

R² is a C₁-C₆ alkyl optionally substituted with halogen, a C₃-C₇ cycloalkyl optionally substituted with halogen, a phenyl optionally substituted with halogen or C₁-C₄alkyl or a C₁-C₄alkyl-phenyl optionally substituted with halogen or C₁-C₄alkyl;

R³ is H or C₁-C₄ alkyl

R² and R³ taken together and the carbon atom they are attached form a C₃-C₇ cycloalkyl;

R⁴ is C₁-C₈ alkyl optionally substituted with halogen or C₁-C₄alkoxy, a C₃-C₇ cycloalkyl optionally substituted with halogen, a phenyl optionally substituted with halogen or C₁-C₄alkyl; a C₁-C₄alkyl-phenyl optionally substituted with halogen or C₁-C₄alkyl or a 4 to 7 membered heterocycle containing 1 to 3 heteroatom selected from N, S, and O, wherein said heterocycle is optionally substituted with one or more halogen, or C₁-C₄ alkyl.

In another embodiment, the invention provides a compound of formula (I), (II) or (III) or a pharmaceutically acceptable salt thereof, wherein:

R¹ is

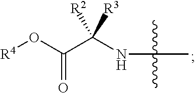

R² is a C₁-C₄ alkyl optionally substituted with halogen, a C₃-C₇ cycloalkyl optionally substituted with halogen, or a C₁-C₄alkyl-phenyl optionally substituted with halogen or C₁-C₄alkyl;

R³ is H or C₁-C₄ alkyl;

R² and R³ taken together and the carbon atom they are attached form a C₃-C₇ cycloalkyl;

R⁴ is C₁-C₈ alkyl optionally substituted with halogen or C₁-C₄alkoxy, a C₃-C₇ cycloalkyl, a C₁-C₄alkyl-phenyl optionally substituted with halogen or C₁-C₄alkyl or a 6 membered heterocycle containing 1 to 3 heteroatom selected from N, S, and O.

In another embodiment, the invention provides a compound of formula (I), (II) or (III) or a pharmaceutically acceptable salt thereof, wherein:

R¹ is

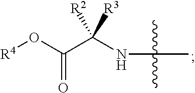

R² is a C₁-C₄ alkyl optionally substituted with halogen;

R³ is H;

R⁴ is C₁-C₈ alkyl optionally substituted with halogen or C₁-C₄alkoxy, a C₃-C₇ cycloalkyl, a C₁-C₄alkyl-phenyl optionally substituted with halogen or C₁-C₄alkyl or a 6 membered heterocycle containing 1 to 3 heteroatom selected from N, S, and O.

In another embodiment, the invention provides a compound of formula (I), (II) or (III) or a pharmaceutically acceptable salt thereof, wherein:

R¹ is selected from the group consisting of

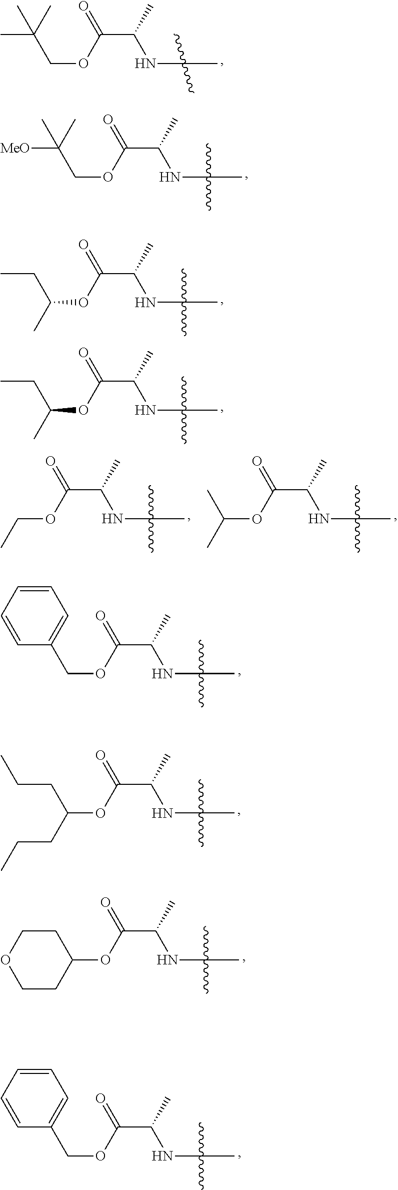

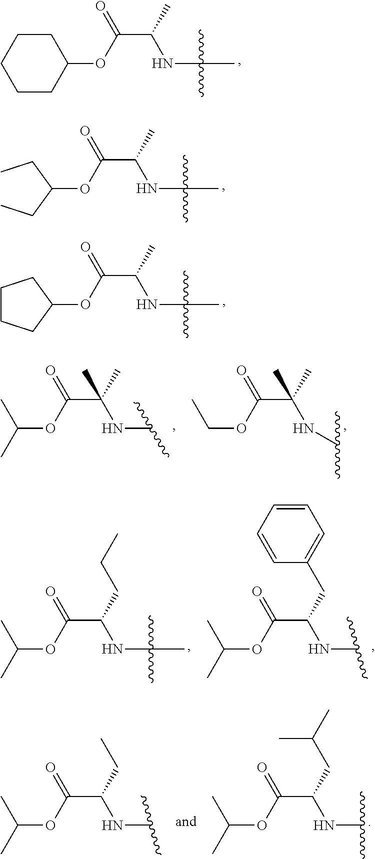

In another embodiment, the invention provides a compound of formula (I), (II) or (III) or a pharmaceutically acceptable salt thereof, wherein:

R¹ is selected from the group consisting of

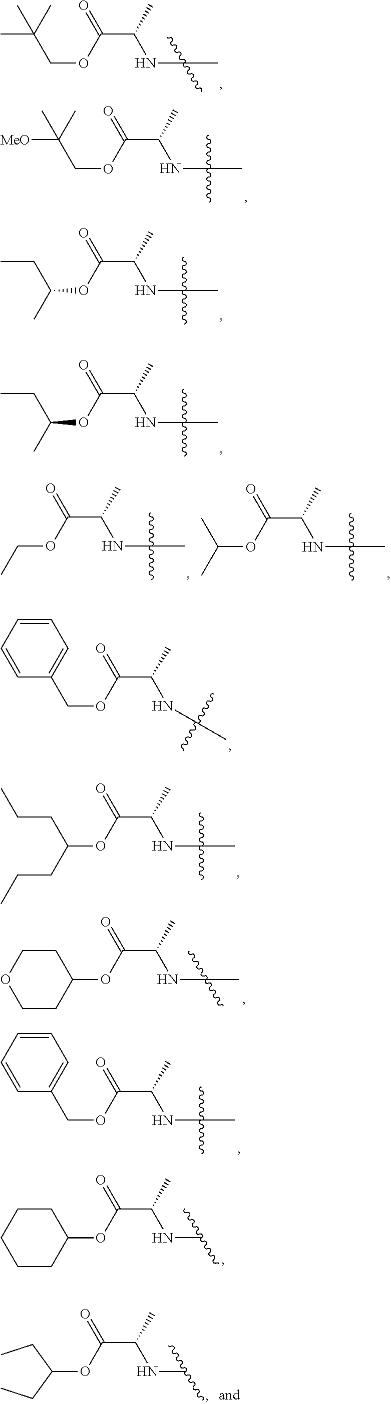

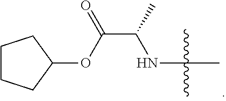 [SMILES: *N[C@@H](C)C(=O)OC1CCCC1]

In another embodiment, the invention provides a compound or pharmaceutically acceptable salt thereof selected from the group consisting of:

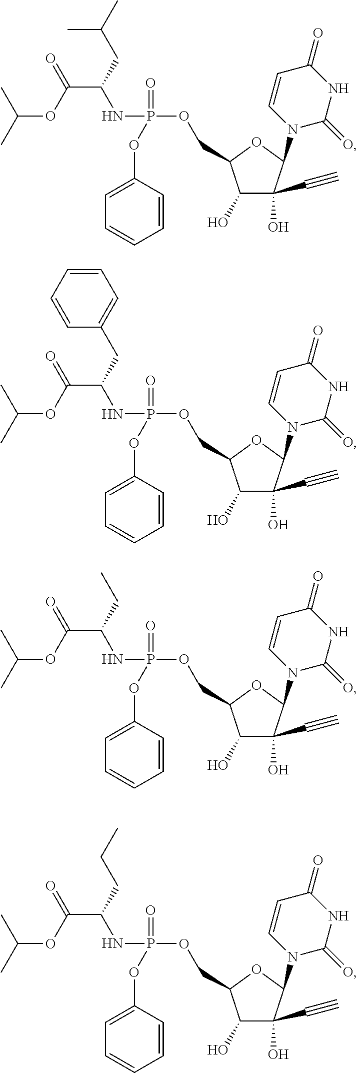

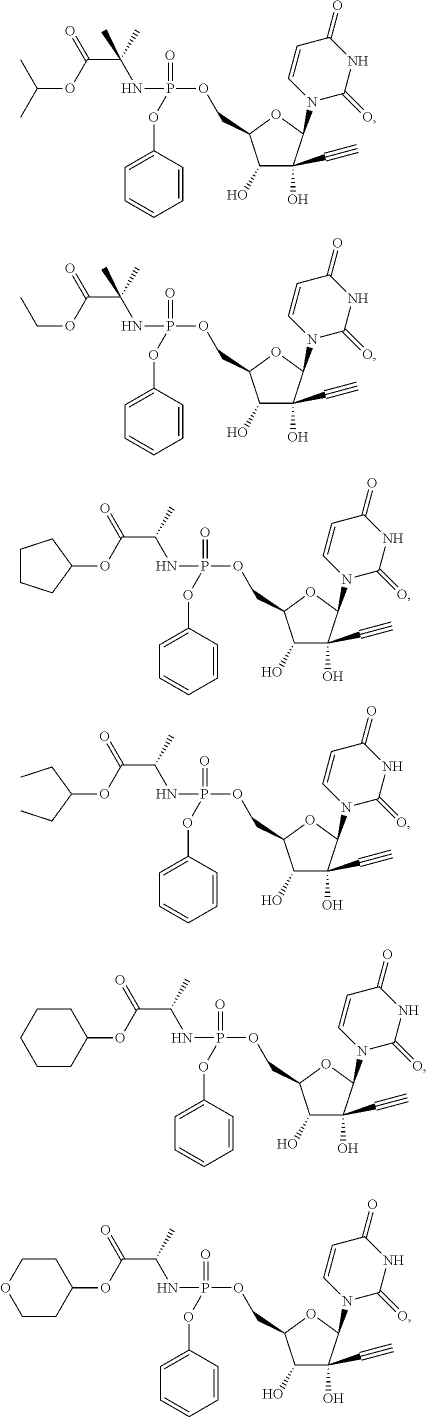

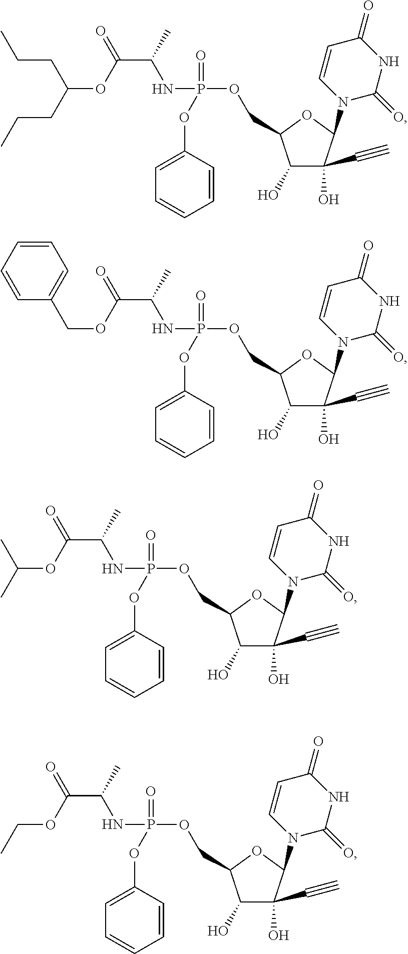

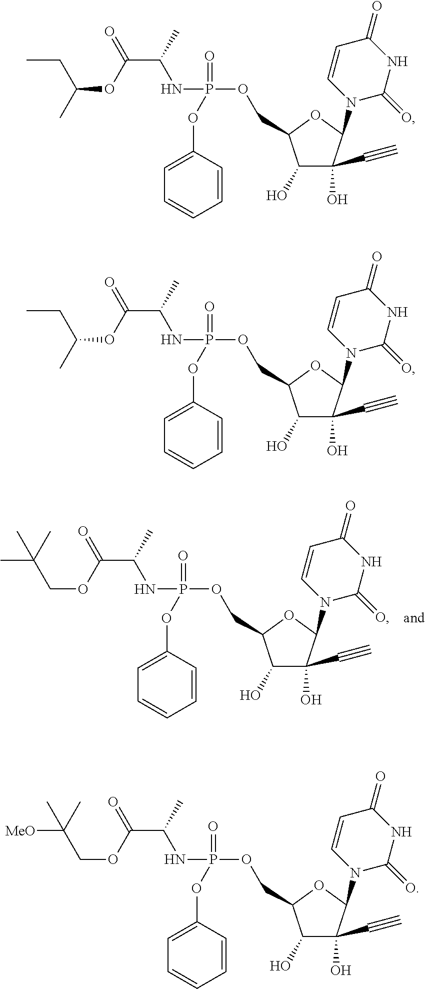 [SMILES: C#C[C@@]1(O)[C@H](O)[C@@H](COP(=O)(N[C@@H](C)C(=O)O[C@@H](C)CC)Oc2ccccc2)O[C@H]1n1ccc(=O)[nH]c1=O ; C#C[C@@]1(O)[C@H](O)[C@@H](COP(=O)(N[C@@H](C)C(=O)O[C@H](C)CC)Oc2ccccc2)O[C@H]1n1ccc(=O)[nH]c1=O ; C#C[C@@]1(O)[C@H](O)[C@@H](COP(=O)(N[C@@H](C)C(=O)OCC(C)(C)C)Oc2ccccc2)O[C@H]1n1ccc(=O)[nH]c1=O ; "and" ; C#C[C@@]1(O)[C@H](O)[C@@H](COP(=O)(N[C@@H](C)C(=O)OCC(C)(C)OC)Oc2ccccc2)O[C@H]1n1ccc(=O)[nH]c1=O]

In another embodiment, the invention provides a compound represented by

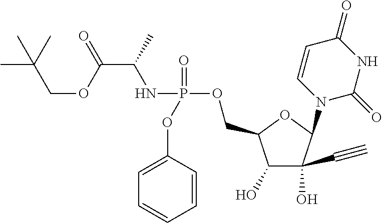 [SMILES: C#C[C@@]1(O)[C@H](O)[C@@H](COP(=O)(N[C@@H](C)C(=O)OCC(C)(C)C)Oc2ccccc2)O[C@H]1n1ccc(=O)[nH]c1=O]

or a pharmaceutically acceptable salt thereof.

In another embodiment, the invention provides a compound represented by

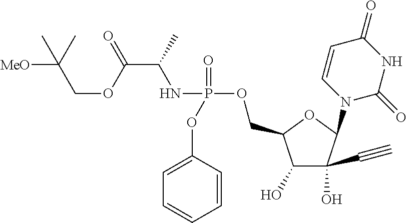 [SMILES: C#C[C@@]1(O)[C@H](O)[C@@H](COP(=O)(N[C@@H](C)C(=O)OCC(C)(C)OC)Oc2ccccc2)O[C@H]1n1ccc(=O)[nH]c1=O]

or a pharmaceutically acceptable salt thereof.

In another embodiment, the invention provides a compound represented by

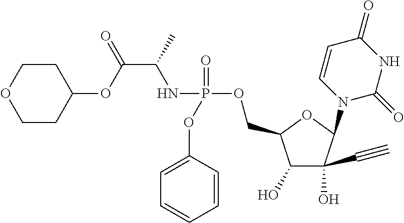 [SMILES: C#C[C@@]1(O)[C@H](O)[C@@H](COP(=O)(N[C@@H](C)C(=O)OC2CCOCC2)Oc2ccccc2)O[C@H]1n1ccc(=O)[nH]c1=O]

or a pharmaceutically acceptable salt thereof.

In another embodiment, the invention provides a compound represented by

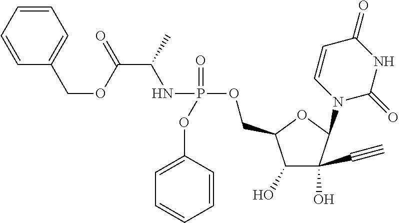 [SMILES: C#C[C@@]1(O)[C@H](O)[C@@H](COP(=O)(N[C@@H](C)C(=O)OCc2ccccc2)Oc2ccccc2)O[C@H]1n1ccc(=O)[nH]c1=O]

or a pharmaceutically acceptable salt thereof.

In another embodiment, the invention provides a compound represented by

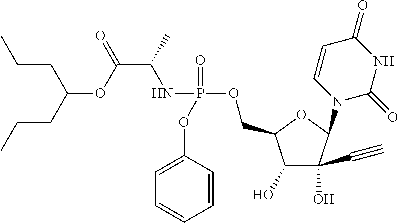 [SMILES: C#C[C@@]1(O)[C@H](O)[C@@H](COP(=O)(N[C@@H](C)C(=O)OC(CCC)CCC)Oc2ccccc2)O[C@H]1n1ccc(=O)[nH]c1=O]

or a pharmaceutically acceptable salt thereof.

In another embodiment, the invention provides a compound represented by

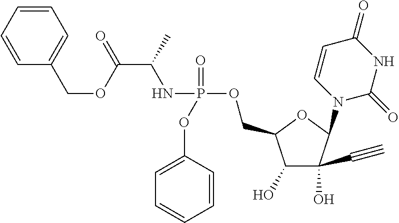 [SMILES: C#C[C@@]1(O)[C@H](O)[C@@H](COP(=O)(N[C@@H](C)C(=O)OCc2ccccc2)Oc2ccccc2)O[C@H]1n1ccc(=O)[nH]c1=O]

or a pharmaceutically acceptable salt thereof.

In another embodiment, the invention provides a compound represented by

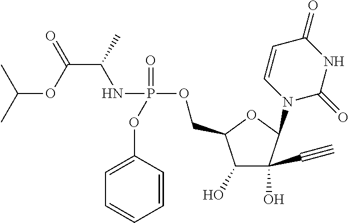 [SMILES: C#C[C@@]1(O)[C@H](O)[C@@H](COP(=O)(N[C@@H](C)C(=O)OC(C)C)Oc2ccccc2)O[C@H]1n1ccc(=O)[nH]c1=O]

or a pharmaceutically acceptable salt thereof.

In another embodiment, the invention provides a compound represented by

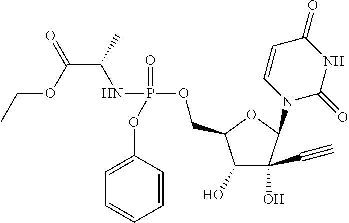 [SMILES: C#C[C@@]1(O)[C@H](O)[C@@H](COP(=O)(N[C@@H](C)C(=O)OCC)Oc2ccccc2)O[C@H]1n1ccc(=O)[nH]c1=O]

or a pharmaceutically acceptable salt thereof.

In another embodiment, the invention provides a compound represented by

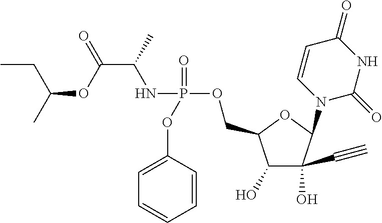 [SMILES: C#C[C@@]1(O)[C@H](O)[C@@H](COP(=O)(N[C@@H](C)C(=O)O[C@@H](C)CC)Oc2ccccc2)O[C@H]1n1ccc(=O)[nH]c1=O]

or a pharmaceutically acceptable salt thereof.

In another embodiment, the invention provides a compound represented by

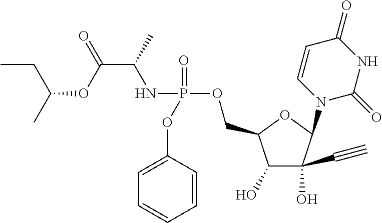 [SMILES: C#C[C@@]1(O)[C@H](O)[C@@H](COP(=O)(N[C@@H](C)C(=O)O[C@H](C)CC)Oc2ccccc2)O[C@H]1n1ccc(=O)[nH]c1=O]

or a pharmaceutically acceptable salt thereof.

In another embodiment, the invention provides a compound represented by

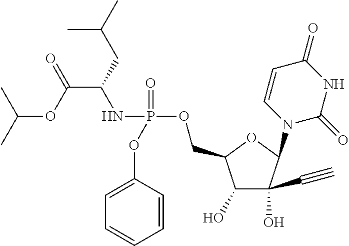 [SMILES: C#C[C@@]1(O)[C@H](O)[C@@H](COP(=O)(N[C@@H](CC(C)C)C(=O)OC(C)C)Oc2ccccc2)O[C@H]1n1ccc(=O)[nH]c1=O]

or a pharmaceutically acceptable salt thereof.

In another embodiment, the invention provides a compound represented by

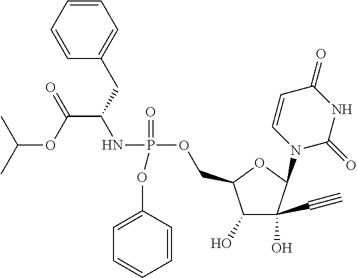 [SMILES: C#C[C@@]1(O)[C@H](O)[C@@H](COP(=O)(N[C@@H](Cc2ccccc2)C(=O)OC(C)C)Oc2ccccc2)O[C@H]1n1ccc(=O)[nH]c1=O]

or a pharmaceutically acceptable salt thereof.

In another embodiment, the invention provides a compound represented by

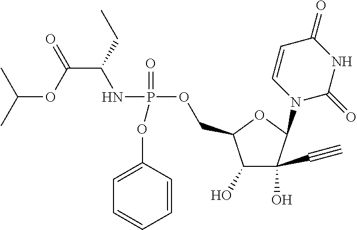 [SMILES: C#C[C@@]1(O)[C@H](O)[C@@H](COP(=O)(N[C@@H](CC)C(=O)OC(C)C)Oc2ccccc2)O[C@H]1n1ccc(=O)[nH]c1=O]

or a pharmaceutically acceptable salt thereof.

In another embodiment, the invention provides a compound represented by

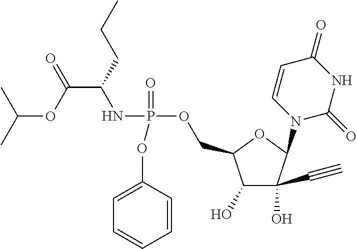 [SMILES: C#C[C@@]1(O)[C@H](O)[C@@H](COP(=O)(N[C@@H](CCC)C(=O)OC(C)C)Oc2ccccc2)O[C@H]1n1ccc(=O)[nH]c1=O]

or a pharmaceutically acceptable salt thereof.

In another embodiment, the invention provides a compound represented by

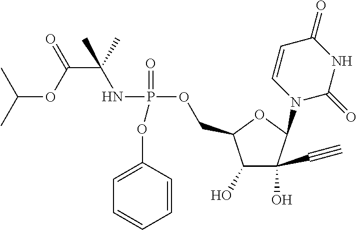

or a pharmaceutically acceptable salt thereof.

In another embodiment, the invention provides a compound represented by

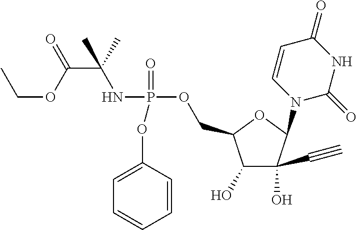

or a pharmaceutically acceptable salt thereof.

In another embodiment, the invention provides a compound represented by

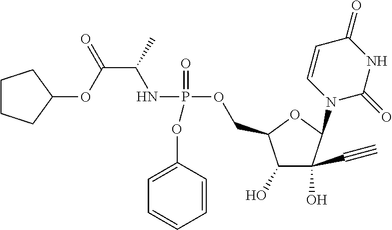 [SMILES: C#C[C@@]1(O)[C@H](O)[C@@H](COP(=O)(N[C@@H](C)C(=O)OC2CCCC2)Oc2ccccc2)O[C@H]1n1ccc(=O)[nH]c1=O]

or a pharmaceutically acceptable salt thereof.

In another embodiment, the invention provides a compound represented by

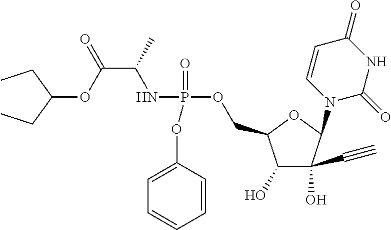 [SMILES: C#C[C@@]1(O)[C@H](O)[C@@H](COP(=O)(N[C@@H](C)C(=O)OC(CC)CC)Oc2ccccc2)O[C@H]1n1ccc(=O)[nH]c1=O]

or a pharmaceutically acceptable salt thereof.

In another embodiment, the invention provides a compound represented by

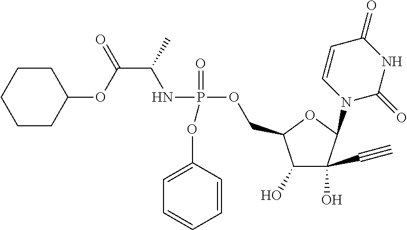 [SMILES: C#C[C@@]1(O)[C@H](O)[C@@H](COP(=O)(N[C@@H](C)C(=O)OC2CCCCC2)Oc2ccccc2)O[C@H]1n1ccc(=O)[nH]c1=O]

or a pharmaceutically acceptable salt thereof

In another embodiment, the invention provides a compound represented by

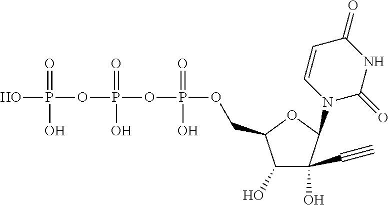 [SMILES: C#C[C@@]1(O)[C@H](O)[C@@H](COP(=O)(O)OP(=O)(O)OP(=O)(O)O)O[C@H]1n1ccc(=O)[nH]c1=O]

or a pharmaceutically acceptable salt.

In another aspect the invention provides a pharmaceutical composition comprising a compound of formula (I), (II) or (III) as defined above, in association with at least one pharmaceutically acceptable excipient, e.g. appropriate diluent and/or carrier, e.g. including fillers, binders, disintegrators, flow conditioners, lubricants, sugars or sweeteners, fragrances, preservatives, stabilizers, wetting agents and/or emulsifiers, solubilisers, salts for regulating osmotic pressure and/or buffers.

In another aspect, the invention provides a compound of formula (I), (II) or (III) as defined above for use as a medicament.

In another aspect the invention provides a compound of formula (I), (II) or (III) for the manufacture of a medicament.

In another aspect the invention provides the use of a compound of formula (I), (II) or (III) for the manufacture of a medicament, e.g. a pharmaceutical composition, for the treatment and/or prevention of a viral infection.

In another aspect the invention provides the use of a compound of formula (I), (II) or (III) as a pharmaceutical, e.g. for the treatment and/or prevention of a viral infection.

In another aspect, the invention provides a compound of formula (I), (II) or (III) as defined above for use in the treatment and/or prevention of a viral infection.

In another aspect, the invention provides the use of a compound of formula (I), (II) or (III) as defined above in the manufacture of a medicament for the treatment and/or prevention of a disease caused by a viral infection.

In still another aspect, the invention provides a method of treating and/or preventing a disease caused by a viral infection, comprising administering to a subject in need thereof an effective amount of a compound of formula (I), (II) or (III) as defined above.

In yet another aspect, the invention provides a pharmaceutical composition for the treatment and/or prevention of a disease caused by a viral infection, comprising a compound of formula (I), (II) or (III) as defined above.

The viral infection is, for example, caused by a virus of the family Flaviviridae, such as dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus, Gadgets Gully virus, Kyasanur Forest disease virus, Langat virus, Louping ill virus, Powassan virus, Royal Farm virus, Karshi virus, Kadam virus, Meaban virus, Saumarez Reef virus, Tyuleniy virus, Aroa virus, Bussuquara vius, Iguape virus, Naranjal virus, Kedougou virus, Cacipacore virus, Koutango virus, Alfuy virus, Usutu virus, Yaounde virus, Kokobera virus, Stratford virus, Bagaza virus, Ilheus virus, Rocio virus, Israeli turkey meningoencephalomyelitis virus, Ntaya virus, Tembusu virus, Sponweni virus, Banzi virus, Bouboui virus, Edge Hill virus, Jugra virus, Potiskum virus, Saboya virus, Sepik virus, Uganda virus, Wesselsbron virus, Entebbe bat virus, Sokoluk virus, Yokose virus, Apoi virus, Cowbone Ridge virus, Jutiapa virus, modoc virus, Sal Vieja virus, San Perlita virus, Bukalasa bat virus, Carey Island virus, Dakar bat virus, Mantana myotis leukoencephalitis virus, Batu Cave virus, Phnom Penh bat virus, Rio Bravo virus, Cell fusing agent virus, Tamana bat virus and Hepatitis C virus, especially dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus.

In yet another aspect, the invention provides a pharmaceutical composition for the treatment and/or prevention of a disease caused by a dengue virus, comprising a compound of formula (I), (II) or (III) as defined above.

In yet another aspect, the invention provides a pharmaceutical composition for the treatment and/or prevention of a disease caused by a Hepatitus C virus, comprising a compound of formula (I), (II) or (III) as defined above.

In another aspect the invention provides a combination of a compound of formula (I), (II) or (III) with at least one second drug substance.

In another embodiment, a pharmaceutical combination composition, comprising:
a therapeutically effective amount of the compound according to any one of above embodiments of Formulae (I) to (III) or a pharmaceutically acceptable salt thereof, and
one or more therapeutically active agents are selected from Interferons, ribavirin and ribavirin analogs, cyclophilin binder, HCV NS3 protease inhibitors, HCV NS5a inhibitors, nucleoside and non-nucleoside NS5b inhibitors, HCV NS4a antagonists, TLR-7 agonists, HCV IRES inhibitors, pharmacokinetic enhancers, anti-fibrotic agents, or mixtures thereof.

In another embodiment, a pharmaceutical combination composition, wherein the one or more therapeutically active agents are selected from Interferons, ribavirin and ribavirin analogs, cyclophilin binder, HCV NS3 protease inhibitors, HCV NS5a inhibitors, nucleoside and non-nucleoside NS5b inhibitors, or mixtures thereof.

In another embodiment, a pharmaceutical combination composition, wherein the one or more therapeutically active agents are selected from ribavirin and ribavirin analogs, cyclophilin binder, HCV NS3 protease inhibitors, HCV NS5a inhibitors, nucleoside and non-nucleoside NS5b inhibitors, or mixtures thereof.

In another embodiment, a pharmaceutical combination composition, wherein the one or more therapeutically active agents are selected from ribavirin and ribavirin analogs, cyclophilin binder, HCV NS3 protease inhibitors, HCV NS5a inhibitors, or mixtures thereof.

In another embodiment, a pharmaceutical combination composition, wherein the one or more therapeutically active agents are selected from ribavirin and ribavirin analogs, cyclophilin binder, HCV NS3 protease inhibitors, or mixtures thereof.

In another embodiment, a pharmaceutical combination composition, wherein the one or more therapeutically active agents are are selected from ribavirin and ribavirin analogs, cyclophilin binder, HCV NS5a inhibitors, or mixtures thereof.

In another embodiment, a pharmaceutical combination composition, wherein the one or more therapeutically active agents are selected from HCV NS3 protease inhibitors, HCV NS5a inhibitors, or mixtures thereof.

In the above methods for using the compounds of the invention, a compound of formula (I), (II) or (III) may be administered to a system comprising cells or tissues. In other embodiments, a compound of formula (I), (II) or (III) may be administered to a human or animal subject.

### DETAILED DESCRIPTION

The compounds of defined above may be synthesized by general synthetic route below, specific examples of which is described in more detail in the Examples.

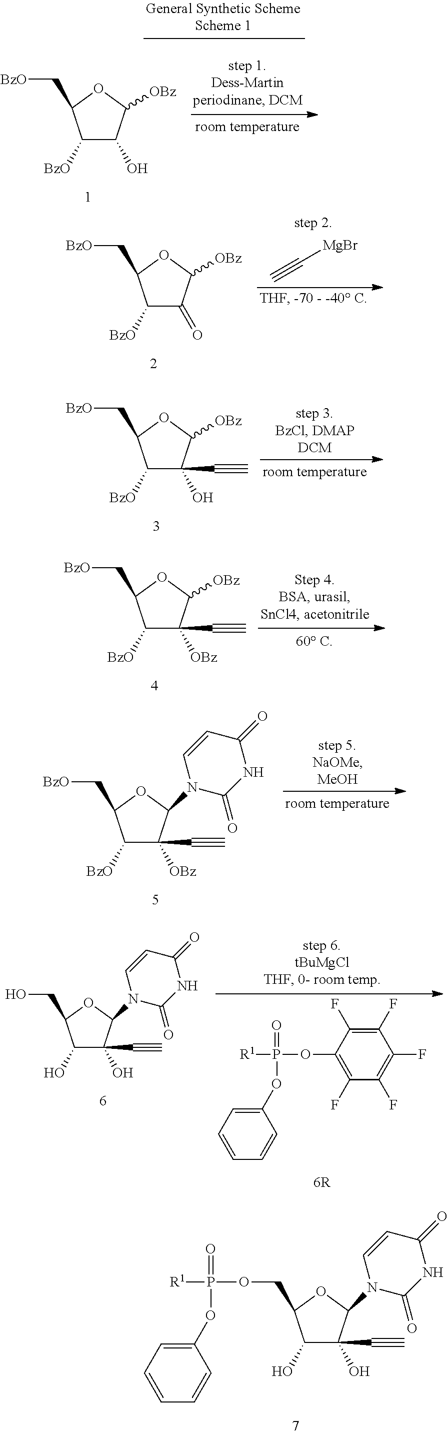

Scheme 1 depicts the synthesis of compound 7 as diastereomers. Compound 7 can be made substantially optically pure by either using substantially optically pure starting material 6R or by separation chromatography.

The compounds of the invention, and particularly as exemplified, in free or pharmaceutically acceptable addition salt form, exhibit pharmacological activity and are useful as pharmaceuticals, particularly for the treatment and/or prevention of viral infections such as those caused by members of the family Flaviviridae. The compounds are particularly useful for the treatment and/or prevention of infections such as those caused by dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus, and other Flaviviridae viruses as described herein.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material.

Compounds of the present invention and intermediates can also be converted into each other according to methods generally known to those skilled in the art.

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/l, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known to those skilled in the art. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

Salts can be converted into the free compounds in accordance with methods known to those skilled in the art. Metal and ammonium salts can be converted, for example, by treatment with suitable acids, and acid addition salts, for example, by treatment with a suitable basic agent.

Mixtures of isomers obtainable according to the invention can be separated in a manner known to those skilled in the art into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g. using chromatographic methods, distribution methods, (re-) crystallization, and the like.

The following applies in general to all processes mentioned herein before and hereinafter.

All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described under “Additional process steps”.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

The compounds of the present invention, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

All starting materials, building blocks, reagents, acids, bases, dehydrating agents, solvents and catalysts utilized to synthesize the compounds of the present invention are either commercially available or can be produced by organic synthesis methods known to one of ordinary skill in the art (Houben-Weyl 4^{th} Ed. 1952, Methods of Organic Synthesis, Thieme, Volume 21).

The term “an optical isomer” or “a stereoisomer” refers to any of the various stereoisomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present invention, including salts, hydrates and solvates thereof, may inherently or by design form polymorphs.

As used herein, the terms “salt” or “salts” refers to an acid addition or base addition salt of a compound of the present invention. “Salts” include in particular “pharmaceutically acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds of the present invention. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds of the present invention, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of subjects. In particular, an ¹⁸F labeled compound of the present invention may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagent in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the present invention. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the present invention that contain groups capable of acting as donors and/or acceptors for hydrogen bonds may be capable of forming co-crystals with suitable co-crystal formers. These co-crystals may be prepared from compounds of the present invention by known co-crystal forming procedures. Such procedures include grinding, heating, co-subliming, co-melting, or contacting in solution compounds of the present invention with the co-crystal former under crystallization conditions and isolating co-crystals thereby formed. Suitable co-crystal formers include those described in WO 2004/078163. Hence the invention further provides co-crystals comprising a compound of the present invention.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease by affecting a viral polymerase RNA chain elogation. In another non-limiting embodiment, the term “a therapeutically effective amount” refers to the amount of the compound of the present invention that, when administered to a cell, or a tissue, or a non-cellular biological material, or a medium, is effective to affect a viruses polymerase RNA chain elogation.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the subject. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

It is therefore indicated that for the treatment of viral infections, such as those caused by a virus of the family Flaviviridae, for example dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus, and other Flaviviridae viruses as described herein, a compound of the invention may be administered to larger mammals, for example humans, by similar modes of administration at similar dosages to those conventionally used.

Moreover, it will be appreciated that the dosage range of a compound of the invention to be employed for treating and/or preventing a viral infection depends upon factors known to the person skilled in the art, including host, nature and severity of the condition to be treated, the mode of administration and the particular substance to be employed.

The daily dosage of the compound of the invention will vary with the compound employed, the mode of administration, the treatment desired and the disease indicated, as well as other factors such as a subject's age, body weight, general health, condition, prior medical history and sex, and like factors known in the medical arts. For example, a compound of the invention is administered at a daily dosage in the range from about 0.5 mg/kg body weight to about 15 mg/kg body weight, e.g. in the range from about 1 mg/kg body weight to about 10 mg/kg body weight. Typically, satisfactory results can be obtained when the compound of the invention is administered at a daily dosage from about 0.001 g to about 1.5 g, e.g. not exceeding about 1 gram, e.g. from about 0.1 g to about 0.5 g for a 70 kg human, given up to 4 times daily.

For pharmaceutical use one or more compounds of the invention may be used, e.g. one, or a combination of two or more compounds of the invention, preferably one compound of the invention, is used.

Depending on the mode of administration, the pharmaceutical composition will preferably comprise from 0.05 to 99.5% by weight, more preferably from 0.1 to 70% by weight, more preferably from 30 to 70% by weight of the active ingredient, and from 0.05 to 99.95% by weight, more preferably from 0.1 to 70% by weight, more preferably from 30 to 70% by weight of a pharmaceutically acceptable carrier, all percentages being based on the total composition.

The pharmaceutical composition may additionally contain various other ingredients known in the art, for example, a lubricant, stabilising agent, buffering agent, emulsifying agent, viscosity-regulating agent, surfactant or preservative.

It is especially advantageous to formulate the pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

As noted above, daily dosages with respect to the second drug substance used will vary depending upon, for example, the compound employed, the host, the mode of administration and the severity of the condition to be treated. For example, lamivudine may be administered at a daily dosage of 100 mg. The pegylated interferon may be administered parenterally one to three times per week, preferably once a week, at a total weekly dose ranging from 2 to 10 million IU, more preferable 5 to 10 million IU, most preferable 8 to 10 million IU. Because of the diverse types of second drug substance that may be used, the amounts can vary greatly, and can be determined by routine experimentation, as described above.

The compound of the invention and a second drug substance may be administered by any conventional route, in particular enterally, e.g. orally, for example in the form of solutions for drinking, tablets or capsules or parenterally, for example in the form of injectable solutions or suspensions.

Conjugates of interferon to a water-soluble polymer are meant to include especially conjugates to polyalkylene oxide homopolymers such as polyethylene glycol (PEG) or polypropylene glycols, polyoxyethylenated polyols, copolymers thereof and block copolymers thereof. As an alternative to polyalkylene oxide-based polymers, effectively non-antigenic materials such as dextran, polyvinyl pyrrolidones, polyacrylamides, polyvinyl alcohols, carbohydrate-based polymers and the like can be used. Such interferon-polymer conjugates are described in U.S. Pat. Nos. 4,766,106, 4,917,888, European Patent Application No. 0 236 987, European Patent Application No. 0 510 356 and International Application Publication No. WO 95/13090, the disclosures of which are. Since the polymeric modification sufficiently reduces antigenic responses, the foreign interferon need not be completely autologous. Interferon used to prepare polymer conjugates may be prepared from a mammalian extract, such as human, ruminant or bovine interferon, or recombinantly produced. Preferred are conjugates of interferon to polyethylene glycol, also known as pegylated interferons.

Especially preferred conjugates of interferon are pegylated alfa-interferons, for example pegylated interferon-α-2a, pegylated interferon-α-2b; pegylated consensus interferon or pegylated purified interferon-α product. Pegylated interferon-α-2a is described e.g. in European Patent 593,868 (incorporated herein by reference in its entirety) and commercially available e. g. under the tradename PEGASYS® (Hoffmann-La Roche). Pegylated interferon-α-2b is described, e.g. in European Patent 975,369 (incorporated herein by reference in its entirety) and commercially available e.g. under the tradename PEG-INTRON A® (Schering Plough). Pegylated consensus interferon is described in WO 96/11953 (incorporated herein by reference in its entirety). The preferred pegylated α-interferons are pegylated interferon-α-2a and pegylated interferon-α-2b. Also preferred is pegylated consensus interferon.

Other preferred second drug substances include fusion proteins of an interferon, for example fusion proteins of interferon-α-2a, interferon-α-2b; consensus interferon or purified interferon-α product, each of which is fused with another protein. Certain preferred fusion proteins comprise an interferon (e.g., interferon-α-2b) and an albumin as described in U.S. Pat. No. 6,973,322 and international publications WO02/60071, WO05/003296 and WO05/077042 (Human Genome Sciences). A preferred interferon conjugated to a human albumin is Albuferon (Human Genome Sciences).

Cyclosporins which bind strongly to cyclophilin but are not immunosuppressive include those cyclosporins recited in U.S. Pat. Nos. 5,767,069 and 5,981,479 and are incorporated herein by reference. [Melle]⁴-cyclosporin is a preferred non-immunosuppressive cyclosporin. Certain other cyclosporin derivatives are described in WO2006039668 (Scynexis) and WO2006038088 (Debiopharm SA) and are incorporated herein by reference. A cyclosporin is considered to be non-immunosuppressive when it has an activity in the Mixed Lymphocyte Reaction (MLR) of no more than 5%, preferably no more than 2%, that of cyclosporin A. The Mixed Lymphocyte Reaction is described by T. Meo in “Immunological Methods”, L. Lefkovits and B. Pens, Eds., Academic Press, N.Y. pp. 227-239 (1979). Spleen cells (0.5×10⁶) from Balb/c mice (female, 8-10 weeks) are co-incubated for 5 days with 0.5×10⁶ irradiated (2000 rads) or mitomycin C treated spleen cells from CBA mice (female, 8-10 weeks). The irradiated allogeneic cells induce a proliferative response in the Balb/c spleen cells which can be measured by labeled precursor incorporation into the DNA. Since the stimulator cells are irradiated (or mitomycin C treated) they do not respond to the Balb/c cells with proliferation but do retain their antigenicity. The IC₅₀ found for the test compound in the MLR is compared with that found for cyclosporin A in a parallel experiment. In addition, non-immunosuppressive cyclosporins lack the capacity of inhibiting CN and the downstream NF-AT pathway. [Melle]⁴-cyclosporin is a preferred non-immunosuppressive cyclophilin-binding cyclosporin for use according to the invention.

Ribavirin (1-β-D-ribofuranosyl-1-1,2,4-triazole-3-caroxamide) is a synthetic, non-interferon-inducing, broad spectrum antiviral nucleoside analog sold under the trade name Virazole (The Merck Index, 11^{th} edition, Editor: Budavar, S, Merck & Co., Inc., Rahway, N.J., p 1304, 1989). U.S. Pat. Nos. 3,798,209 and RE29,835 disclose and claim ribavirin. Ribavirin is structurally similar to guanosine, and has in vitro activity against several DNA and RNA viruses including Flaviviridae (Gary L. Davis, Gastroenterology 118:S104-S114, 2000).

Other combinations include those of a compound of the invention with a non-immunosuppressive cyclophilin-binding cyclosporine, with mycophenolic acid, a salt or a prodrug thereof, and/or with a S1P receptor agonist, e.g. FTY720.

Additional examples of second drug substances that can be used in combination with a compound of the invention include:

(1) Interferons, including interferon alpha 2a or 2b and pegylated (PEG) interferon alpha 2a or 2b, for example:
(a) Intron-A®, interferon alfa-2b (Schering Corporation, Kenilworth, N.J.);
(b) PEG-Intron®, peginteferon alfa-2b (Schering Corporation, Kenilworth, N.J.);
(c) Roferon®, recombinant interferon alfa-2a (Hoffmann-La Roche, Nutley, N.J.);
(d) Pegasys®, peginterferon alfa-2a (Hoffmann-La Roche, Nutley, N.J.);
(e) Berefor®, interferon alfa 2 available (Boehringer Ingelheim Pharmaceutical, Inc., Ridgefield, Conn.);
(f) Sumiferon®, a purified blend of natural alpha interferons (Sumitomo, Japan)
(g) Wellferon®, lymphoblastoid interferon alpha n1 (GlaxoSmithKline);
(h) Infergen®, consensus alpha interferon (InterMune Pharmaceuticals, Inc., Brisbane, Calif.);
(i) Alferon®, a mixture of natural alpha interferons (Interferon Sciences, and Purdue Frederick Co., CT);

### (j) Viraferon®;

(k) Consensus alpha interferon from Amgen, Inc., Newbury Park, Calif.

Other forms of interferon include: interferon beta, gamma, tau and omega, such as Rebif (Interferon beta 1a) by Serono, Omniferon (natural interferon) by Viragen, REBIF (interferon beta-1a) by Ares-Serono, Omega Interferon by BioMedicines; oral Interferon Alpha by Amarillo Biosciences; an interferon conjugated to a water soluble polymer or to a human albumin, e.g., Albuferon (Human Genome Sciences), an antiviral agent, a consensus interferon, ovine or bovine interferon-tau.

Conjugates of interferon to a water-soluble polymer are meant to include especially conjugates to polyalkylene oxide homopolymers such as polyethylene glocol (PEG) or polypropylene glycols, polyoxyethylenated polyols, copolymers thereof and block copolymers thereof. As an alternative to polyalkylene oxid-based polymers, effectively non-antigenic materials such as dextran, polyvinyl pyrrolidones, polyacrylamides, polyvinyl alcohols, carbohydrate-based polymers and the like can be used. Since the polymeric modification sufficiently reduces antigenic response, the foreign interferon need not be completely autologous. Interferon used to prepare polymer conjugates may be prepared from a mammalian extract, such as human, ruminant or bovine interferon, or recombinantly produced. Preferred are conjugates of interferon to polyethylene glycol, also known as pegylated interferons.

(2) Ribavirin, such as ribavirin (1-beta-D-ribofuranosyl-1H-1,2,4-triazole-3-carboxamide) from Valeant Pharmaceuticals, Inc., Costa Mesa, Calif.); Rebetol® from Schering Corporation, Kenilworth, N.J., and Copegus® from Hoffmann-La Roche, Nutley, N.J.; and new ribavirin analogues in development such as Levovirin and Viramidine by Valeant.
(3) Thiazolidine derivatives which show relevant inhibition in a reverse-phase HPLC assay with an NS3/4A fusion protein and NS5A/5B substrate (Sudo K. et al., Antiviral Research, 1996, 32, 9-18), especially compound RD-1-6250, possessing a fused cinnamoyl moiety substituted with a long alkyl chain, RD4 6205 and RD4 6193.
(4) Thiazolidines and benzanilides identified in Kakiuchi N. et al. J. FEBS Letters 421, 217-220; Takeshita N. et al. Analytical Biochemistry, 1997, 247, 242-246.
(5) A phenanthrenequinone possessing activity against protease in a SDS-PAGE and autoradiography assay isolated from the fermentation culture broth of Streptomyces sp., Sch 68631 (Chu M. et al., Tetrahedron Letters, 1996, 37, 7229-7232), and Sch 351633, isolated from the fungus Penicillium griseofulvum, which demonstrates activity in a scintillation proximity assay (Chu M. et al, Bioorganic and Medicinal Chemistry Letters 9, 1949-1952).
(6) Protease inhibitors; examples include substrate-based NS3 protease inhibitors (Attwood et al., Antiviral peptide derivatives, PCT WO 98/22496, 1998; Attwood et al., Antiviral Chemistry and Chemotherapy 1999, 10, 259-273; Attwood et al, Preparation and use of amino acid derivatives as anti-viral agents, German Patent Pub. DE 19914474; Tung et al. Inhibitors of serine proteases, particularly hepatitis C virus NS3 protease; PCT WO 98/17679), including alphaketoamides and hydrazinoureas, and inhibitors that terminate in an electrophile such as a boronic acid or phosphonate (Llinas-Brunet et al. Hepatitis C inhibitor peptide analogues, PCT WO 99/07734) are being investigated.

Non-substrate-based NS3 protease inhibitors such as 2,4,6-trihydroxy-3-nitro-benzamide derivatives (Sudo K. et al., Biochemiscal and Biophysical Research Communications, 1997, 238 643-647; Sudo K. et al. Antiviral Chemistry and Chemotherapy, 1998, 9, 186), including RD3-4082 and RD3-4078, the former substituted on the amide with a 14 carbon chain and the latter processing a para-phenoxyphenyl group are also being investigated.

Sch 68631, a phenanthrenequinone, is an Hepatitis C virus protease inhibitor (Chu M et al., Tetrahedron Letters 37:7229-7232, 1996). In another example by the same authors, Sch 351633, isolated from the fungus Penicillium grieofulvum, was identified as a protease inhibitor (Chu M. et al., Bioorganic and Medicinal Chemistry Letters 9:1949-1952). Nanomolar potency against the Hepatitis C virus NS3 protease enzyme has been achieved by the design of selective inhibitors based on the macromolecule eglin c. Eglin c, isolated from leech, is a potent inhibitor of several serine proteases such as S. griseus proteases A and B, α-chymotrypsin, chymase and subtilisin. Qasim M. A. et al., Biochemistry 36:1598-1607, 1997.

U.S. patents disclosing protease inhibitors for the treatment of Hepatitis C virus include, for example, U.S. Pat. No. 6,004,933 to Spruce et al (incorporated herein by reference in its entirety) which discloses a class of cysteine protease inhibitors for inhibiting Hepatitis C virus endopeptidase 2; U.S. Pat. No. 5,990,276 to Zhang et al. (incorporated herein by reference in its entirety) which discloses synthetic inhibitors of hepatitis C virus NS3 protease; U.S. Pat. No. 5,538,865 to Reyes et al. (incorporated herein by reference in its entirety). Peptides as NS3 serine protease inhibitors of Hepatitis C virus are disclosed in WO 02/008251 to Corvas International, Inc., and WO 02/08187 and WO 02/008256 to Schering Corporation. Hepatitis C virus inhibitor tripeptides are disclosed in U.S. Pat. Nos. 6,534,523, 6,410,531 and 6,420,380 to Boehringer Ingelheim and WO 02/060926 to Bristol Myers Squibb. Diaryl peptides as NS3 serine protease inhibitors of Hepatitis C virus are disclosed in WO 02/48172 to Schering Corporation (incorporated herein by reference). Imidazoleidinones as NS3 serine protease inhibitors of Hepatitis C virus are disclosed in WO 02/18198 to Schering Corporation and WO 02/48157 to Bristol Myers Squibb. WO 98/17679 to Vertex Pharmaceuticals and WO 02/48116 to Bristol Myers Squibb also disclose Hepatitis C virus protease inhibitors.

Hepatitis C virus NS3-4A serine protease inhibitors including BILN 2061 by Boehringer Ingelheim, Telaprevir (VX-950) by Vertex, boceprivir by Merck, and other compounds currently in preclinical development.

Substrate-based NS3 protease inhibitors, including alphaketoamides and hydrazinoureas, and inhibitors that terminate in an elecrophile such as a boronic acid or phosphonate; Non-substrate-based NS3 protease inhibitors such as 2,4,6-trihydroxy-3-nitro-benzamide derivatives including RD3-4082 and RD3-4078, the former substituted on the amide with a 14 carbon chain and the latter processing a para-phenoxyphenyl group; and Sch68631, a phenanthrenequinone, an Hepatitis C virus protease inhibitor.

Sch 351633, isolated from the fungus Penicillium griseofulvum was identified as a protease inhibitor. Eglin c, isolated from leech is a potent inhibitor of several serine proteases such as S. griseus proteases A and B, α-chymotrypsin, chymase and subtilisin.

U.S. Pat. No. 6,004,933 discloses a class of cysteine protease inhibitors from inhibiting Hepatitis C virus endopeptidase 2; synthetic inhibitors of Hepatitis C virus NS3 protease; Hepatitis C virus inhibitor tripeptides; diaryl peptides such as NS3 serine protease inhibitors of Hepatitis C virus; imidazolidindiones as NS3 serine protease inhibitors of Hepatitis C virus.

Thiazolidines and benzanilides. Thiazolidine derivatives which show relevant inhibition in a reverse-phase HPLC assay with an NS3/4A fusion protein and NS5A/5B substrate especially compound RD-16250 possessing a fused cinnamoyl moiety substituted with a long alkyl chain, RD4 6205 and RD4 6193

Phenanthrenequinone possessing activity against protease in a SDS-PAGE and autoradiography assay isolated from the fermentation culture broth of Streptomyces sp, Sch68631 and Sch351633, isolated from the fungus Penicillium griseofulvum, which demonstrates activity in a scintillation proximity assay.

(7) Nucleoside or non-nucleoside inhibitors of Hepatitis C virus NS5B RNA-dependent RNA polymerase, such as 2′-C-methyl-3′-O-L-valine ester ribofuranosyl cytidine (Idenix) as disclosed in WO 2004/002422 A2, R803 (Rigel), JTK-003 (Japan Tabacco), HCV-086 (ViroPharma/Wyeth) and other compounds currently in development; gliotoxin and the natural product cerulenin; 2′-fluoronucleosides; other nucleoside analogues as disclosed in WO 02/057287 A2, WO 02/057425 A2, WO 01/90121, WO 01/92282, and U.S. Pat. No. 6,812,219.

Idenix Pharmaceuticals discloses the use of branched nucleosides in the treatment of flaviviruses (including Hepatitis C virus) and pestiviruses in International Publication Nos. WO 01/90121 and WO 01/92282. Specifically, a method for the treatment of hepatitis C infection (and flaviviruses and pestiviruses) in humans and other host animals is disclosed in the Idenix publications that includes administering an effective amount of a biologically active 1′, 2′, 3′ or 4′-branched β-D or β-L nucleosides or a pharmaceutically acceptable salt or prodrug thereof, administered either alone or in combination with another antiviral agent, optionally in a pharmaceutically acceptable carrier. Certain preferred biologically active 1′, 2′, 3′, or 4′ branched β-D or β-L nucleosides, including Telbivudine, are described in U.S. Pat. Nos. 6,395,716 and 6,875,751.

Other patent applications disclosing the use of certain nucleoside analogs to treat hepatitis C virus include: PCTCA00/01316 (WO 01/32153; filed Nov. 3, 2000) and PCT/CA01/00197 (WO 01/60315; filed Feb. 19, 2001) filed by BioChem Pharma, Inc., (now Shire Biochem, Inc.); PCT/US02/01531 (WO 02/057425; filed Jan. 18, 2002) and PCT/US02/03086 (WO 02/057287; filed Jan. 18, 2002) filed by Merck & Co., Inc., PCT/EP01/09633 (WO 02/18404; published Aug. 21, 2001) filed by Roche, and PCT Publication Nos. WO 01/79246 (filed Apr. 13, 2001), WO 02/32920 (filed Oct. 18, 2001) and WO 02/48165 by Pharmasset, Ltd.

PCT Publication No. WO 99/43691 to Emory University, entitled “2′-Fluoronucleosides” discloses the use of certain 2′-fluoronucleosides to treat Hepatitis C virus.

Eldrup et al. (Oral Session V, Hepatitis C Virus, Flaviviridae; 16^{th} International Conference on Antiviral Research (Apr. 27, 2003, Savannah, Ga.)) describes the structure activity relationship of 2′-modified nucleosides for inhibition of Hepatitis C virus.

Bhat et al. (Oral Session V, Hepatitis C Virus, Flaviviridae, 2003 (Oral Session V, Hepatitis C Virus, Flaviviridae; 16^{th} International conference on Antiviral Research (Apr. 27, 2003, Savannah, Ga.); p A75) describes the synthesis and pharmacokinetic properties of nucleoside analogues as possible inhibitors of Hepatitis C virus RNA replication. The authors report that 2′-modified nucleosides demonstrate potent inhibitory activity in cell-based replicon assays.

Olsen et al. (Oral Session V, Hepatitis C Virus, Flaviviridae; 16^{th} International Conference on Antiviral Research (Apr. 27, 2003, Savannah, Ga.)p A76) also describe the effects of the 2′-modified nucleosides on Hepatitis C virus RNA replication.

(8) Nucleotide polymerase inhibitors and gliotoxin (Ferrari R. et al. Journal of Virology, 1999, 73, 1649-1654), and the natural product cerulenin (Lohmann V. et al. Virology, 1998, 249, 108-118).
(9) Hepatitis C virus NS3 helicase inhibitors, such as VP-50406 by ViroPhama and compounds from Vertex. Other helicase inhibitors (Diana G. D. et al., Compounds, compositions and methods for treatment of hepatitis C, U.S. Pat. No. 5,633,358 (incorporated herein by reference in its entirety); Diana G. D. et al., Piperidine derivatives, pharmaceutical compositions thereof and their use in the treatment of hepatitis C, WO 97/36554).
(10) Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the Hepatitis C virus RNA (Alt M. et al., Archives of Virology, 1997, 142, 589-599; Galderisi U. et al., Journal of Cellular Physiology, 199, 181, 251-257); such as ISIS 14803 by Isis Pharm/Elan, antisense by Hybridon, antisense by AVI bioPharma.
(11) Inhibitors of IRES-dependent translation (Ikeda N et al., Agent for the prevention and treatment of hepatitis C, Japanese Patent Pub. JP-08268890; Kai Y et al. Prevention and treatment of viral diseases, Japanese Patent Pub. JP-10101591); such as ISIS 14803 by Isis Pharm/Elan, IRES inhibitor by Anadys, IRES inhibitors by Immusol, targeted RNA chemistry by PTC Therapeutics.
(12) Ribozymes, such as nuclease-resistant ribozymes (Maccjak, D. J. et al., Hepatology 1999, 30, abstract 995) and those directed in U.S. Pat. No. 6,043,077 to Barber et al., and U.S. Pat. Nos. 5,869,253 and 5,610,054 to Draper et al. for example, HEPTAZYME by RPI.
(13) siRNA directed against Hepatitis C virus genome.
(14) Hepatitis C virus replication inhibitor of any other mechanisms such as by VP50406ViroPharama/Wyeth, inhibitors from Achillion, Arrow.
(15) An inhibitor of other targets in the Hepatitis C virus life cycle including viral entry, assembly and maturation.
(16) An immune modulating agent such as an IMPDH inhibitor, mycophenolic acid, a salt or a prodrug thereof sodium mycophenolate or mycophenolate mofetil, or Merimebodib (VX-497); thymosin alpha-1 (Zadaxin, by SciClone); or a S1P receptor agonist, e.g. FTY720 or analogue thereof optionally phosphorylated.
(17) An anti-fibrotic agent, such as a N-phenyl-2-pyrimidine-amine derivative, imatinib (Glivec), IP-501 by Indevus, and Interferon gamma 1b from InterMune.
(18) Therapeutic vaccine by Intercell, Epimmune/Genecor, Merix, Tripep (Chron-VacC), immunotherapy (Therapore) by Avant, T cell therapy by CellExSys, monoclonal antibody XTL-002 by STL, ANA 246 and ANA 246 BY Anadys.
(19) Other miscellaneous compounds including 1-amino-alkylcyclohexanes (U.S. Pat. No. 6,034,134 to Gold et al.), alkyl lipids (U.S. Pat. No. 5,922,757 to Chojkier et al.), vitamin E and other antitoxidants (U.S. Pat. No. 5,922,757 to Chojkier et al.), amantadine, bile acids (U.S. Pat. No. 5,846,99964 to Ozeki et al.), N-(phosphonoacetl)-L-aspartic acid,) U.S. Pat. No. 5,830,905 to Diana et al.), benzenedicarboxamides (U.S. Pat. No. 5,633,388 to Diane et al.), polyadenylic acid derivatives (U.S. Pat. No. 5,496,546 to Wang et al.), 2′3′-dideoxyinosine (U.S. Pat. No. 5,026,687 to Yarchoan et al.), benzimidazoles (U.S. Pat. No. 5,891,874 to Colacino et al.), plant extracts (U.S. Pat. No. 5,837,257 to Tsai et al., U.S. Pat. No. 5,725,859 to Omer et al., and U.S. Pat. No. 6,056,961) and piperidines (U.S. Pat. No. 5,830,905 to Diana et al.). Also, squalene, telbivudine, N-(phosphonoacetyl)-L-aspartic acid, benzenedicarboxamides, polyadenylic acid derivatives, glycosylation inhibitors, and nonspecific cytoprotective agents that block cell injury caused by the virus infection.
(20) Any other compound currently in preclinical or clinical development for the treatment of Hepatitis C virus, including Interleukin-10 (Schering-Plough), AMANTADINE (Symmetrel) by Endo Labs Solvay, caspase inhibitor IDN-6556 by Idun Pharma, HCV/MF59 by Chiron, CIVACIR (Hepatitis C Immune Globulin) by NABI, CEPLENE (histamine dichloride) by Maxim, IDN-6556 by Idun PHARM, T67, a beta-tubulin inhibitor, by Tularik, a therapeutic vaccine directed to E2 by Innogenetics, FK788 by Fujisawa Helathcare, IdB1016 (Siliphos, oral silybin-phosphatidyl choline phytosome), fusion inhibitor by Trimeris, Dication by Immtech, hemopurifier by Aethlon Medical, UT 231B by United Therapeutics.
(21) Purine nucleoside analog antagonists of TIR7 (toll-like receptors) developed by Anadys, e.g., Isotorabine (ANA245) and its prodrug (ANA975), which are described in European applications EP348446 and EP636372, International Publications WO03/045968, WO05/121162 and WO05/25583, and U.S. Pat. No. 6,973,322.
(21) Non-nucleoside inhibitors developed by Genelabs and described in International Publications WO2004/108687, WO2005/12288, and WO2006/076529.
(22) Other second drug substances (e.g., non-immunomodulatory or immunomodulatory compounds) that may be used in combination with a compound of this invention include, but are not limited to, those specified in WO 02/18369.

In another aspect, this invention provides a method comprising administering a compound of the invention and anoher anti-viral agent, preferably an anti-Flaviviridae, e.g. and anti-dengue or anti-Hepatitis C virus agent. Such anti-viral agents include, but are not limited to, immunomodulatory agents, such as α, β, and δ interferons, pegylated derivatized interferon-α compounds, and thymosin; other anti-viral agents, such as ribavirin, amantadine, and telbivudine; other inhibitors of hepatitis C proteases (NS2-NS3 inhibitors and NS3-NS4A inhibitors); inhibitors of other targets in the Flaviviridae (e.g. dengue virus, Hepatitis C virus) life cycle, including helicase, polymerase, and metalloprotease inhibitors; inhibitors of internal ribosome entry; broad-spectrum viral inhibitors, such as IMPDH inhibitors (e.g., compounds of U.S. Pat. Nos. 5,807,876, 6,498,178, 6,344,465, 6,054,472, WO 97/40028, WO 98/40381, WO 00/56331, and mycophenolic acid and derivatives thereof, and including, but not limited to VX-497, VX-148, and/or VX-944); or combinations of any of the above.

Each component of a combination according to this invention may be administered separately, together, or in any combination thereof. As recognized by skilled practitioners, dosages of interferon are typically measured in IU (e.g., about 4 million IU to about 12 million IU). Each component may be administered in one or more dosage forms. Each dosage form may be administered to the subject in any order.

### EXAMPLES

The invention is described with reference to the following examples. It is to be appreciated that the invention is not limited to these examples.

### ABBREVIATIONS

DMSO dimethylsulfoxide
THF tetrahydrofuran
DMAP 4-dimethylaminopyridine
NMR nuclear magnetic resonance
TEA triethylamine
MS mass spectroscopy
DMF dimethylformamide
DCM dichloromethane
PBS phosphate buffered saline
FBS fetal bovine serum
HRP horse radish peroxidase
TMB 3,3′,5,5′-tetramethylbenzidine
DMEM Dulbecco's Modified Eagle's Medium

### I. Preparation of Compounds of the Invention

### Example 1

### Synthesis of (S)-isopropyl 2-(((S)-(((2R,3R,4R,5R)-5-(2,4-dioxo-3,4-dihydropyrimidin-1(2H)-yl)-4-ethynyl-3,4-dihydroxytetrahydrofuran-2 yl)methoxy)(phenoxy)phosphoryl)amino)propanoate

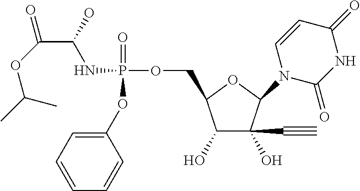 [SMILES: C#C[C@@]1(O)[C@H](O)[C@@H](CO[P@@](=O)(N[C@@H](O)C(=O)OC(C)C)Oc2ccccc2)O[C@H]1n1ccc(=O)[nH]c1=O]

### Step 1: Synthesis of (4R,5R)-5-((benzoyloxy)methyl)-3-oxotetrahydrofuran-2,4-diyl dibenzoate

To a stirring mixture of Dess-Martin periodinane (1.83 g, 4.32 mmol, 2 equiv.) in DCM (4 ml) at 0° C. was added a solution of (3R,4S,5R)-5-((benzoyloxy)methyl)-3-hydroxytetrahydrofuran-2,4-diyl dibenzoate (1.0 g, 2.16 mmol, 1 equiv.) in DCM (3 ml). The mixture was allowed to warm to room temperature and stirred for 24 h. The solvent was removed in vacuo and the residue was triturated with diethyl ether. The mixture was filtered through a pad of magnesium sulfate and the filtrate was stirred with an equal volume of sodium thiosulfate in saturated sodium bicarbonate for around 10 min (until organic layer appeared clear). The organic layer was separated, washed with brine, dried over anhydrous magnesium sulfate, and concentrated in vacuo to yield the crude material. Upon flash chromatography over silica gel with eluting solvent of hexane/ethyl acetate (7:3), the title compound (0.432 g, 0.938 mmol, 43.4%) was isolated. 1H NMR (300 MHz, CDCl₃): δ 8.13-8.00 (6H, m), 7.62-7.35 (9H, m), 6.21 (1H, s), 5.90 (1H, d), 5.07-4.62 (3H, m).

### Step 2: Synthesis of (3R,4R,5R)-5-((benzoyloxy)methyl)-3-ethynyl-3-hydroxytetrahydrofuran-2,4-diyldibenzoate

To a stirring solution of 0.5M ethynyl magnesium bromide in THF (7.5 ml, 3.75 mmol, 4.0 equiv.) at −78° C., was added a solution of the compound obtained from step 1 (0.432 g, 0.938 mmol, 1 equiv.) in THF (3.0 ml). The mixture was allowed to stir at below −78° C. for 2 h, −40° C. for 1 h. Then, saturated ammonium chloride solution was added at 0° C., and the mixture was allowed to warm to room temperature slowly, stirring for another 1 h. The mixture was extracted twice with ethyl acetate. The combined organic layer was washed with brine, dried over anhydrous magnesium sulfate, and evaporated in vacuo to yield the crude material. The crude title compound was used directly in subsequent reaction without further purification.

### Step 3: Synthesis of (3R,4R,5R)-5-((benzoyloxy)methyl)-3-ethynyltetrahydrofuran-2,3,4-triyl tribenzoate

To a stirring solution of DMAP (0.105 g, 0.938 mmol, 1 equiv.) and triethylamine (0.9 ml) in DCM (9 ml, 6.5 mmol, 6.9 equiv.) was added benzoyl chloride (0.33 ml, 2.81 mmol, 3.0 equiv.). Then, a solution of compound obtained from step 2 (0.938 mmol, 1 equiv.) in DCM (3 ml) was added drop-wise. The mixture was allowed to stir at room temperature for 12 h. The mixture was diluted with DCM, washed with HCl (2N), saturated sodium bicarbonate and brine, dried over anhydrous magnesium sulfate, and evaporated in vacuo to yield the crude material. Upon flash chromatography over silica gel with eluting solvent of hexane/ethyl acetate (90:10), followed by hexane/ethyl acetate (85:15), the title compound (0.224 g, 0.380 mmol, 40.5%) was obtained. 1H NMR (300 MHz, CDCl₃): δ 8.17-7.12 (20H), 6.99 (1H, s), 6.37 (1H), 4.81-4.55 (3H, m), 2.76 (1H, s).

### Step 4: Synthesis of (2R,3R,4R,5R)-5-((benzoyloxy)methyl)-2-(2,4-dioxo-3,4-dihydropyrimidin-1(2H)-yl)-3-ethynyltetrahydrofuran-3,4-diyldibenzoate

To a solution of uracil (56.9 mg, 0.508 mmol) in dry acetonitrile (1 ml) was added BSA (0.502 ml, 2.032 mmol) and the resultant solution was heated at 80° C. to reflux for 1 hr under argon atmosphere. The silylated uracil (56.9 mg, 0.508 mmol) thus obtained was cooled to 0° C. and treated with a solution of compound obtained from step 3 (300 mg, 0.508 mmol) in acetonitrile (2 ml), followed by dropwise addition of tin (IV) chloride (0.208 ml, 1.778 mmol). Then the reaction mixture was warmed to room temperature, then heated at 60° C. for 3 hr. The reaction mixture was poured into ice cold water. The aqueous layer was back extracted with ethyl acetate (20 ml×3). The combined organic layers were washed with brine and dried over sodium sulfate, filtered and concentrated to give crude material. The crude product was purified by silica gel chromatography using hexane/ethyl acetate as eluent. The pure fractions were combined and concentrated in vacuo to give the title compound (125 mg, 0.194 mmol, 38.1% yield) as a brownish paste. 1H NMR (400 MHz, CDCl₃) δ ppm 1.27 (t, J=7.15 Hz, 2H) 2.06 (s, 2H) 2.78 (s, 1H) 4.14 (q, J=7.03 Hz, 1H) 4.64 (dt, J=6.53, 3.51 Hz, 1H) 4.89-5.03 (m, 1H) 5.78 (dd, J=8.28, 2.01 Hz, 1H) 6.07 (d, J=3.01 Hz, 1H) 6.70 (s, 1H) 7.23-7.30 (m, 2H) 7.42-7.55 (m, 4H) 7.56-7.65 (m, 2H) 7.79-7.87 (m, 2H) 8.09 (dd, J=8.41, 1.13 Hz, 2H) 8.12-8.20 (m, 2H) 9.15 (s, 1H). MS (m+1)=581.18; MS (m−1)=579.11.

### Step 5: Synthesis of 1-((2R,3R,4R,5R)-3-ethynyl-3,4-dihydroxy-5-(hydroxymethyl)tetrahydrofuran-2-yl)pyrimidine-2,4(1H,3H)-dione

To a stirred solution of compound obtained from step 4 (125 mg, 0.215 mmol) in methanol (1 ml) was added sodium methoxide 30% (w/w) in methanol (0.121 ml, 2.153 mmol) dropwise at 0° C. Then the mixture was warmed to room temperature and stirred for 1.5 hr. The reaction mixture was neutralized to pH 4 with formic acid at 0° C. The reaction mixture was then concentrated under reduced pressure to give the crude material. The resulting residue was purified by silica gel chromatography using DCM/MeOH as eluents. The pure fractions were combined and concentrated in vacuo to give the title compound (50 mg, 0.166 mmol, 77% yield) as off white solid. 1H NMR (400 MHz, CD₃OD) δ ppm 3.03 (s, 1H) 3.75-3.84 (m, 1H) 3.91-4.01 (m, 2H) 4.22 (d, J=8.78 Hz, 1H) 5.74 (d, J=8.03 Hz, 1H) 6.04 (s, 1H) 8.05 (d, J=8.03 Hz, 1H). MS (m+1)=268.88; MS (m−1)=266.81.

### Step 6: (S)-isopropyl 2-(((S)-(((2R,3R,4R,5R)-5-(2,4-dioxo-3,4-dihydropyrimidin-1(2H)-yl)-4-ethynyl-3,4-dihydroxytetrahydrofuran-2-yl)methoxy)(phenoxy)phosphoryl)amino)propanoate

To a stirred solution of compound obtained from step 5 (146 mg, 0.544 mmol) in THF (1 ml) was added 1.0M tBuMgCl in THF (1.633 ml, 0.544 mmol) dropwise at room temperature. The mixture was stirred at room temperature for 1 hr. Then to the reaction mixture was added with a solution of (S)-isopropyl 2-(((S)-(perfluorophenoxy)(phenoxy)phosphoryl)amino)propanoate (740 mg, 1.633 mmol) in THF (2 ml) dropwise at room temperature. The reaction mixture was then stirred at room temperature for an overnight. Without any workup, the reaction mixture was quenched with 1 ml of water. The mixture was filtered through microfilter via a syringe to give a clear solution and was directly purified. The crude material was purified by HPLC using 20-95% ACN 40 min run method. The pure fractions were combined and lyophilized to give the title compound (40 mg, 0.074 mmol, 13.67% yield), as white solid. 1H NMR (400 MHz, CD₃OD) δ ppm 1.18-1.25 (m, 6H) 1.35 (d, J=7.03 Hz, 3H) 3.08 (s, 1H) 3.86-3.97 (m, 1H) 4.04-4.12 (m, 1H) 4.13-4.19 (m, 1H) 4.36 (ddd, J=11.73, 6.09, 3.76 Hz, 1H) 4.49 (ddd, J=11.80, 6.02, 2.01 Hz, 1H) 4.97 (dt, J=12.55, 6.27 Hz, 1H) 5.61 (d, J=8.28 Hz, 1H) 6.03 (s, 1H) 7.18-7.24 (m, 1H) 7.27 (d, J=8.78 Hz, 2H) 7.34-7.42 (m, 2H) 7.65 (d, J=8.03 Hz, 1H). 31P NMR showed desired product at 3.77 ppm (indicating as single diastereomer assigned as Sp). MS (m+1)=537.96; MS (m−1)=536.15.

### Preparation of (S)-isopropyl 2-(((S)-(perfluorophenoxy)(phenoxy)phosphoryl)amino)propanoate

This material was synthetized according to published procedures. (J. Org. Chem. 2011, 76, 8311-8319)

Other analogs are synthetized in a similar manner as described above.

### II. Antiviral Activity of Compounds of the Invention

### Example 2

### Degue Virus

### 1) Detection of Compounds Antiviral Activity in HuH7 Dengue Replicon Assay (Ref.1)

Test Plates: 96-well plates

Cell line: HuH7-Dengue replicon

Media: DMEM-PRF+2% FCS+1% P/S+2 mM L-Glutamine+0.1 mM NEAA+1 mM SP,

Drug control: Drug stock at 10 mM in 90% DMSO

Incubation: 2 days @ 37° C., 5% CO₂
Day 1: Seed HuH7-Dengue replicon cell suspension 80 μl (1.875×10⁵ cells/ml) in DMEM-PRF+2% FCS+1% P/S+2 mM L-Glutamine+0.1 mM NEAA+1 mM SP, O/N @ 37° C., 5% CO₂
Day 2: Prepare of compounds solution:
   1 μl compounds (from 90% DMSO stock of different dilution prepared in stock plates)+19 μl media in V-bottom 96-well plate.
   Add 20 μl of compounds solution into the replicon cells, incubate for 48 hrs @ 37° C., 5 CO₂
Day 4: Plate for luciferase detection & cell viability detection

### Detection:

From 37° C., add 25 μl of 25 μM ViviRen (Promega) diluted in media (final concentration @ 5 μM), shake, incubate for 20′.
Measure luminescence in Clarity plate reader @ 0.1 s
Add 25 μl of CellTiter-Glo (Promega) (10 μl of CellTiter-Glo+15 μl of media/well), shake for 2 min, leave for 15 min. (keep in dark)
Measure luminescence in Clarity 4.0 plate reader @ 0.1 s

### Solutions:

Media:
   For maintain cells: DMEM (high glucose)+10% FCS+1% P/S+2 mM L-Glutamine+0.1 mM NEAA+10 μg/ml Puromycin
   For assay: DMEM-PRF+2% FCS+1% P/S+2 mM L-Glutamine+0.1 mM NEAA+1 mM SP

### 3) Protocols for Automated 4 Days CCK8 (Dojindo) Cytotoxicity Assay in HepG2 and THP-1 Cells

1. 25 μL of HepG2 suspension containing 1.6×10⁴ cells/ml (400 cells/well) or 25 μL of THP-1 suspension containing 8×10⁴ cells/ml (2000 cells/well) was dispensed into a clear 384-well plate by GNF dispenser 2B vertical-head.
2. The plate was pre-incubated for 24 h in the GNF incubator (humidified, 37° C., 5% CO₂).
3. Serial-diluted compound were directly transferred into the culture media in the plate (200× dilution).
4. Plates were incubated for 96 hours in the GNF incubator.
5. CCK-8 was thawed on the bench-top, and was pre-diluted with media (2.5× dilutions).
6. 35 μL of 2.5× diluted CCK8 was added to each well of the plate by GNF dispenser 1C angled-head.
7. The plates were incubated for 3 hours in the incubator.
8. The absorbance at 450 nm was measured by Envision.

### Dengue Assay on Cryopreserved PBMC Cells

Cryopreserved human PBMC cells were purchased from approved vendors. It was then thawed according to manufacturer's instructions and suspended in RPMI medium supplemented with 1% penicillin/streptomycin solution and 10% Fetal Calf serum. The cells were then counted and viability checked (viability should be at least 70%). After centrifuging and removing the media, the cells were diluted to 1×10⁷ cells/mL in RPMI medium supplemented with 1% penicillin/streptomycin. 50 μl of the cells were then dispensed into 96-well tissue culture plate constituting 5×10⁵ cells/well. Next, virus with humanized 4G2 mixture was prepared for infection. Briefly, virus (2×10⁷ pfu/ml) was mixed with humanized 4G2 antibody with the final antibody concentration of 0.38 μg/ml and incubated for 30 minutes at 4° C. to assist virus/antibody complex formation. The virus-antibody complex was then added to the PBMC at multiplicity of infection (M.O.I) of 0.5. The resulting media was then and further incubated the plates at 37° C. in the humidified incubator for infection to take place. Serial diluted compounds were then added and finally media was added such that the final media volume was 200 μl with 2% Fetal Calf Serum. The plates were then incubated at 37° C., 5% CO₂ for another 48 hours. The extent of the infection and compound inhibition was measured by plaque reduction assay using BHK cells. Briefly, BHK cells grown in 24-well tissue culture were subjected to supernatants derived from infection containing serial diluted compounds. After additional 4 days, the monolayer of BHK cells were fixed, stained with crystal violet stain and plaques counted. Dose response curves were plotted from the mean absorbance (n=3) versus the log of the concentration of test compounds. The EC₅₀ is calculated by nonlinear regression analysis. A positive control (7-deaza-2′-C-acetylene-adenosine) was used to ensure the quality of the data.

### Example 3

### Cytotoxicity with THP-1 Cells

### 4 Day Cytotoxicity Assay Using THP-1 Cells

THP-1 cells grown in suspension were counted and diluted to 8×10⁴ cells/ml in RPMI-1640 media supplemented with 10% fetal bovine serum and 1% penicillin/streptomycin. 25 ul of the THP-1 containing media consisting of 2000 cells were dispensed in 384-well tissue culture plate and pre-incubated at room temperature for 30 minutes, followed by 37° C., 5% CO₂ overnight in the humidified incubator. On the next day, serial-diluted compound plates were prepared and 125 nl of compounds at various concentrations were then dispensed into the tissue culture well (200× dilution). The plates were then transferred to 37° C., 5% CO₂ humidified incubator for additional 96 hours. The plates were then transferred to 37° C., 5% CO₂ humidified incubator for additional 96 hours. Cytotoxicity was measured by CCK-8 assay. Briefly, CCK-8 was thawed on bench top and diluted 2.5× with the growth media. 35 ul of the pre-diluted CCK-8 was then introduced into each well and the plates were then further incubated in 37° C., 5% CO₂ humidified incubator for 3 hours. The absorbance was read by Envision at 450 nm. Dose response curves were plotted from the mean absorbance (n=2) versus the log of the concentration of test compounds. The EC₅₀ is calculated by nonlinear regression analysis. A positive control (puromycin) was used to ensure the quality of the data.

### Example 4

### HCV Replicon Assay

### HCV Replicon Antiviral EC50 Assays.

Replicon cells, expressing the GT1b HCV luciferase replicon, were seeded in 96-well plates at a density of 5,000 cells per well in 100 μl of DMEM culture medium. Compounds were serially diluted in 100% dimethyl sulfoxide (DMSO) and added to cells at a 1:200 dilution at a final concentration of 0.5% DMSO in a total volume of 200 μl. In 96-well assays, 3-fold serial drug dilutions with 11 concentrations were used and the starting concentration was 50 uM. Cell plates were incubated at 37° C. for 3 days, after which culture medium was removed and cells were assayed for luciferase activity as markers for replicon levels. Luciferase expression was quantified using a commercial luciferase assay. Luciferase levels were converted into percentages relative to the levels in the untreated controls defined as 100%.

### HCV Replicon Cytotoxicity CC50 Assay.

Replicon cells, expressing the GT1b HCV luciferase replicon, were seeded in 96-well plates at a density of 5,000 cells per well in 100 μl of DMEM culture medium. Compounds were serially diluted in 100% dimethyl sulfoxide (DMSO) and added to cells at a 1:200 dilution at a final concentration of 0.5% DMSO in a total volume of 200 μl. In 96-well assays, 3-fold serial drug dilutions with 11 concentrations were used and the starting concentration was 50 uM. Cell plates were incubated at 37° C. for 3 days, after which culture medium was removed and cells were assayed for ATP levels, as an indication of proliferation relative to cytotoxic control Staurosporine. ATP levels were quantified by Cell Titer Glow, a commercial luciferase luminescence assay. Luminescence levels were converted into percentages relative to the levels in the untreated controls defined as 100%.

TABLE 1
Dengue, HCV and Cytotoxicity data

| Compound | Dengue | HCV 1b | HCV 1b | THP-1 |
|---|---|---|---|---|
| number | EC₅₀ (μm) | EC₅₀ (μm) | CC₅₀ (μm) | CC50 (μm) |
| 1 | 0.23 | 0.098 | >20 | >50 |

It is understood that the examples and embodiments described herein are for illustrative purposes only and that various modifications or changes in light thereof will be suggested to persons skilled in the art and are to be included within the spirit and purview of this application and scope of the appended claims.

1. A compound of formula I, or a pharmaceutically acceptable salt thereof: 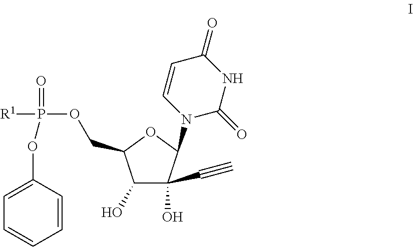
wherein
R¹ is 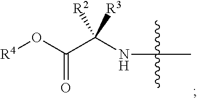
R² is a C₁-C₆ alkyl optionally substituted with halogen, a C₃-C₇ cycloalkyl optionally substituted with halogen, a phenyl optionally substituted with halogen or C₁-C₄alkyl or a C₁-C₄alkyl-phenyl optionally substituted with halogen or C₁-C₄alkyl;
R³ is H or C₁-C₄ alkyl
R² and R³ taken together and the carbon atom they are attached form a C₃-C₇ cycloalkyl;
R⁴ is C₁-C₈ alkyl optionally substituted with halogen or C₁-C₄alkoxy, a C₃-C₇ cycloalkyl optionally substituted with halogen, a phenyl optionally substituted with halogen or C₁-C₄alkyl; a C₁-C₄alkyl-phenyl optionally substituted with halogen or C₁-C₄alkyl or a 4 to 7 membered heterocycle containing 1 to 3 heteroatom selected from N, S, and O, wherein said heterocycle is optionally substituted with one or more halogen, or C₁-C₄ alkyl.

2. The compound according to claim 1, of formula (I), or a pharmaceutically acceptable salt thereof: 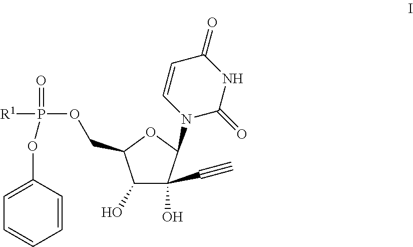
wherein:
R¹ is selected from the group consisting of

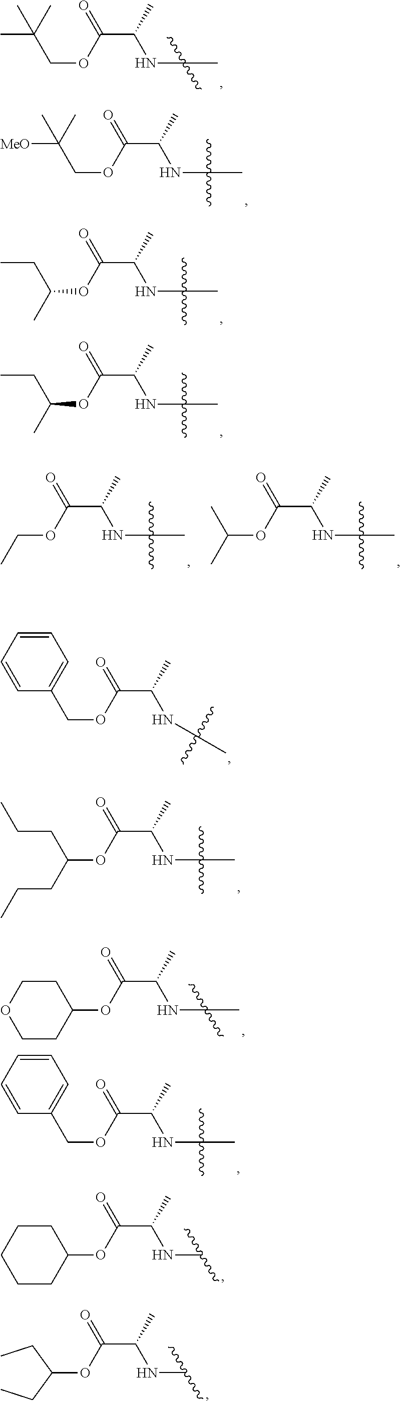

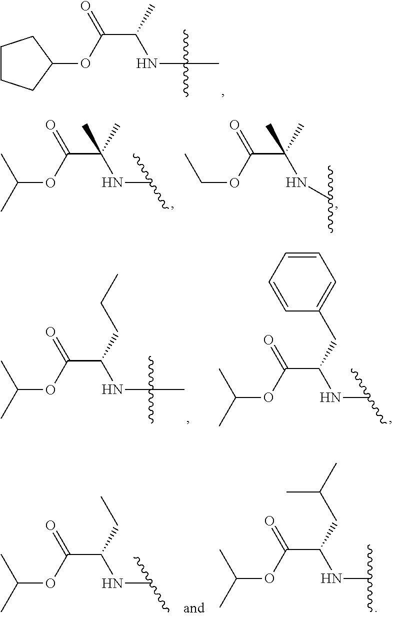

3. The compound according to claim 1, which is a compound of formula (II), or a pharmaceutically acceptable salt thereof: 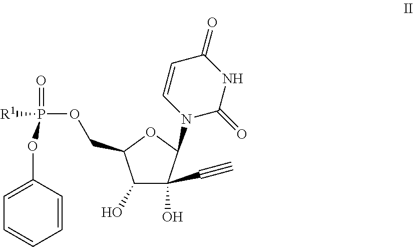
wherein:
R¹ is selected from the group consisting of

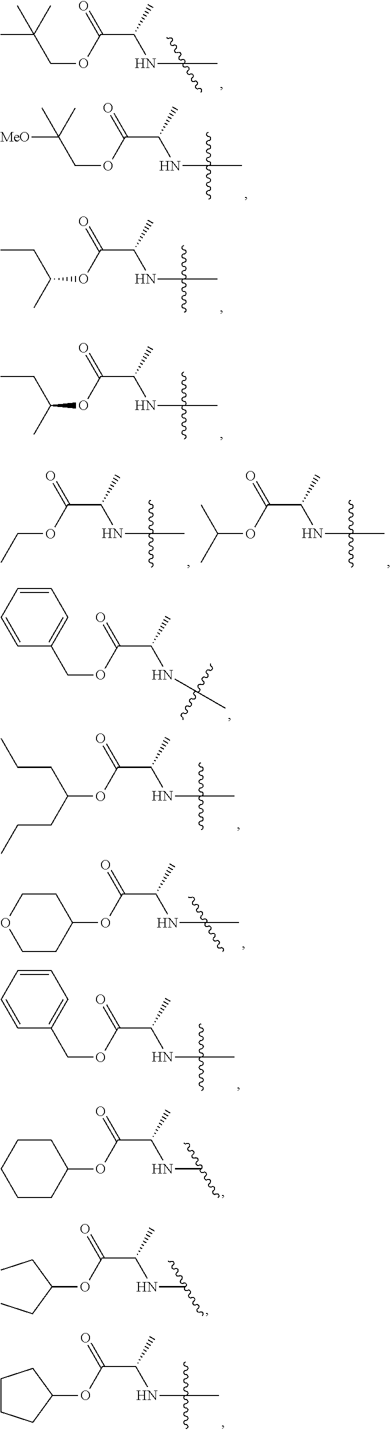

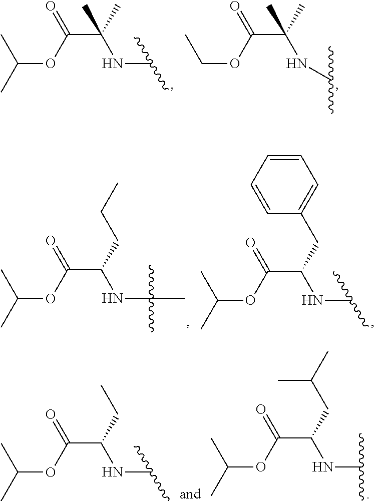

4. The compound according to claim 2, wherein R¹ is selected from the group consisting of

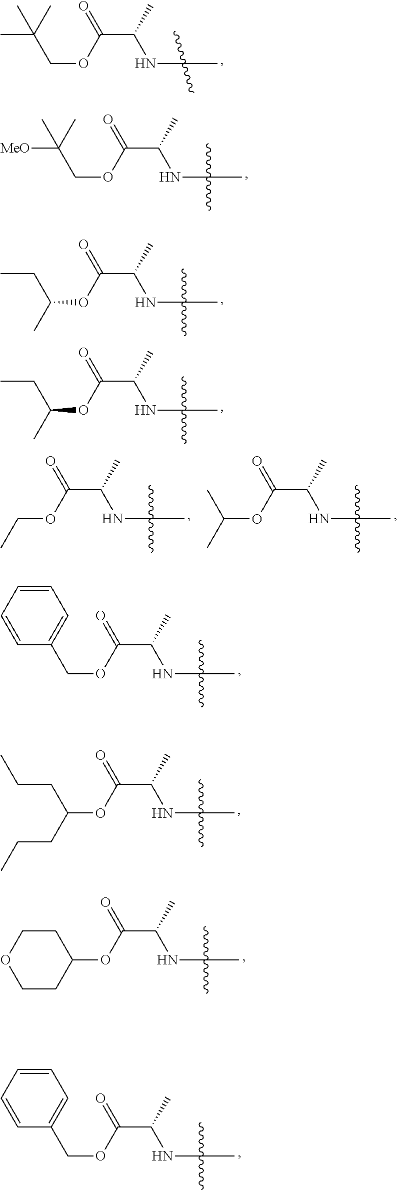

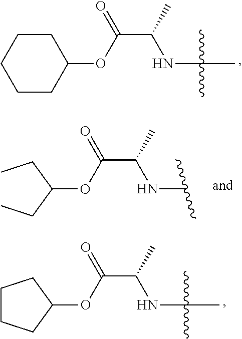 [SMILES: *N[C@@H](C)C(=O)OC1CCCCC1 ; *N[C@@H](C)C(=O)OC(CC)CC ; *N[C@@H](C)C(=O)OC1CCCC1]

5. The compound according to claim 1, or pharmaceutically acceptable salt thereof, wherein the compound is selected from the group consisting of:

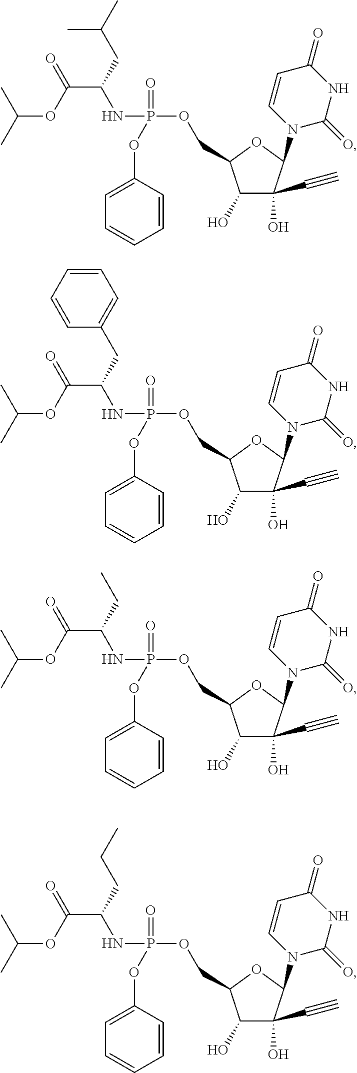

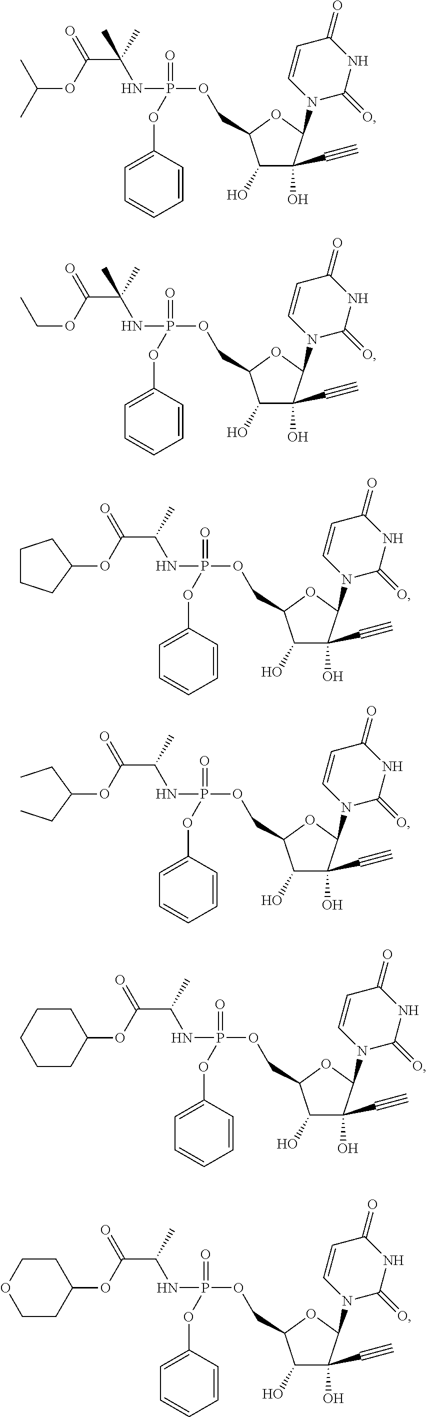

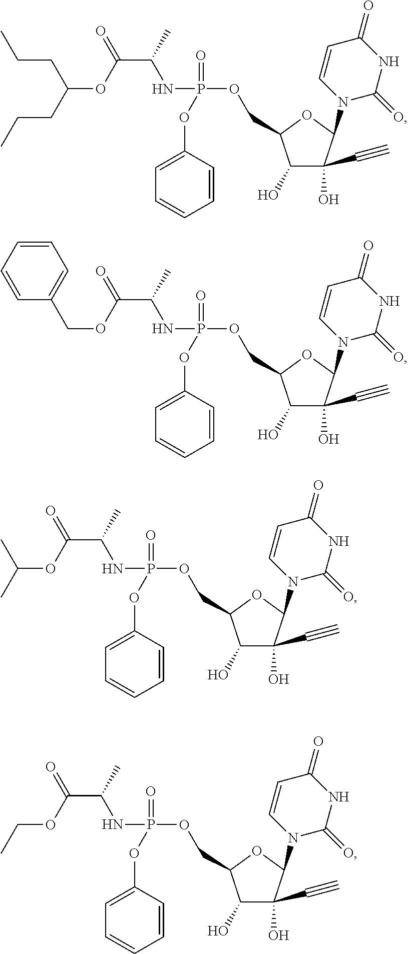

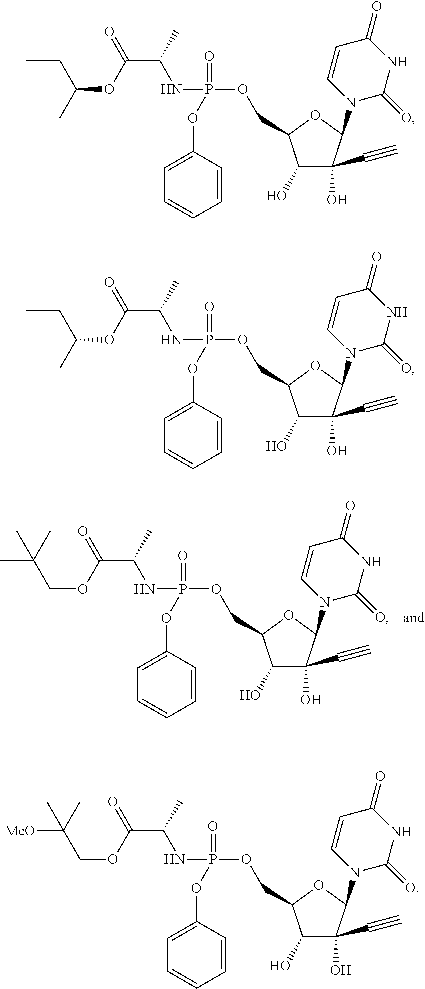 [SMILES: C#C[C@@]1(O)[C@H](O)[C@@H](COP(=O)(N[C@@H](C)C(=O)O[C@@H](C)CC)Oc2ccccc2)O[C@H]1n1ccc(=O)[nH]c1=O ; C#C[C@@]1(O)[C@H](O)[C@@H](COP(=O)(N[C@@H](C)C(=O)O[C@H](C)CC)Oc2ccccc2)O[C@H]1n1ccc(=O)[nH]c1=O ; C#C[C@@]1(O)[C@H](O)[C@@H](COP(=O)(N[C@@H](C)C(=O)OCC(C)(C)C)Oc2ccccc2)O[C@H]1n1ccc(=O)[nH]c1=O ; "and" ; C#C[C@@]1(O)[C@H](O)[C@@H](COP(=O)(N[C@@H](C)C(=O)OCC(C)(C)OC)Oc2ccccc2)O[C@H]1n1ccc(=O)[nH]c1=O]

6. The compound according to claim 1, or pharmaceutically acceptable salt thereof, represented by 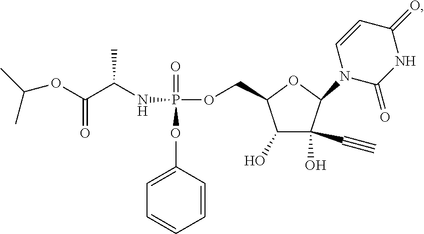 [SMILES: C#C[C@@]1(O)[C@H](O)[C@@H](CO[P@@](=O)(N[C@@H](C)C(=O)OC(C)C)Oc2ccccc2)O[C@H]1n1ccc(=O)[nH]c1=O]
(S)-isopropyl 2-(((S)-(((2R,3R,4R,5R)-5-(2,4-dioxo-3,4-dihydropyrimidin-1(2H)-yl)-4-ethynyl-3,4-dihydroxytetrahydrofuran-2-yl)methoxy)(phenoxy)phosphoryl)amino)propanoate.

7. A pharmaceutical composition, comprising:
the compound as claimed in claim 1, or a pharmaceutically acceptable salt thereof, and
a pharmaceutically acceptable excipient, diluent or carrier.

8. The pharmaceutical composition of claim 7, wherein the compound is a compound according to claim 6.

9. A method of treating and/or preventing a disease caused by a viral infection, comprising:
administering to a subject in need thereof an effective amount of the compound of claim 1.

10. The method according to claim 9, wherein the viral infection is caused by a virus selected from the group consisting of dengue virus, yellow fever virus, West Nile virus, Japanese encephalitis virus, tick-borne encephalitis virus, Kunjin virus, Murray Valley encephalitis, St Louis encephalitis, Omsk hemorrhagic fever virus, bovine viral diarrhea virus, Zika virus and Hepatitis C virus.

11. The method according to claim 9, wherein the viral infection is caused by Hepatitis C virus.

12. The method of claim 9, wherein the compound is a compound according to claim 6.

13. A pharmaceutical combination composition, comprising:
the compound according to claim 1 and
one or more therapeutically active agents.

14. The pharmaceutical combination composition of claim 13, wherein the one or more therapeutically active agents are selected from Interferons, ribavirin and ribavirin analogs, cyclophilin binder, HCV NS3 protease inhibitors, HCV NS5a inhibitors, nucleoside and non-nucleoside NS5b inhibitors, or mixtures thereof.
